(19) United States
(12) Patent Application Publication
(10) Pub. No.: US 2009/0004112 A1
(43) Pub. Date: Jan. 1, 2009

(54) METHODS FOR THE TREATMENT OF NEURODEGENERATIVE DISEASES USING NMDA RECEPTOR GLYCINE SITE ANTAGONISTS

(75) Inventor: Asa ABELIOVICH, New York, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 12/049,782

(22) Filed: Mar. 17, 2008

Related U.S. Application Data
(63) Continuation-in-part of application No. PCT/US2007/009736, filed on Apr. 20, 2007.
(60) Provisional application No. 60/794,003, filed on Apr. 21, 2006.
(60) Provisional application No. 60/853,231, filed on Oct. 20, 2006.
(60) Provisional application No. 60/955,971, filed on Aug. 15, 2007.

(51) Int. Cl. A61K 49/00; A61K 31/47; A61K 31/55; A61K 31/5415; C12Q 1/68; A61P 25/00; C12N 5/00; A61K 31/5025; A61K 31/404; C12Q 1/48

(52) U.S. Cl. 424/9.2; 514/312; 514/313; 514/213.01; 514/223.2; 514/248; 514/419; 435/15; 435/6; 435/375

(57) ABSTRACT

The disclosure provides methods for treating a neurodegenerative disease by administering a NMDA receptor glycine site antagonist. Compounds that can be used in the methods are also provided. Methods are also provided for determining whether a compound inhibits activity of a Parkinson's Disease-associated mutant of leucine-rich repeat kinase-2 (LRRK2). The methods include assessing accumulation of axonal spheroid inclusions, branching and length of neuronal processes, and neuronal cell death.

This application is a continuation-in-part of International Application No. PCT/US2007/009736 filed on Apr. 20, 2007 which claims priority to U.S. Provisional Application No. 60/794,003 filed on Apr. 21, 2006 and U.S. Provisional Application No. 60/853,231 filed on Oct. 20, 2006, this application also claims priority to U.S. Provisional Application No. 60/955,971 filed on Aug. 15, 2007; all of which are hereby incorporated by reference in their entireties.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entireties. The disclosures of these publications in their entireties are hereby incorporated by reference into this application in order to more fully describe the state of the art as known to those skilled therein as of the date of the invention described herein.

### BACKGROUND

Glutamate is the principal excitatory neurotransmitter in the brain. Glutamatergic overstimulation may result in neuronal damage, a phenomenon called excitotoxicity. Such excitotoxicity ultimately leads to neuronal calcium overload, and has been implicated in neurodegenerative disorders. Glutamate stimulates a number of postsynaptic receptors including the N-methyl-D-aspartate (NMDA) receptor, which has been implicated in memory processes, dementia and in the pathogenesis of neurodegenerative disorders like Alzheimer's disease (AD). Accumulating evidence suggests that excitoxicity may also be a mechanism underlying neurodegeneration in Parkinson's disease (PD) as well as in other neurodegenerative diseases such as amyotrophic lateral sclerosis (ALS). Prevalence of PD and AD increases with age and is associated with chronic, progressive and debilitating conditions. There is impairment of higher mental function, with loss of memory as the cardinal symptom. Aphasia, that is loss of ability to use words; agnosia, that is inability to recognize familiar objects; and apraxia, that is inability to execute complex coordinated movements are some of the symptoms which are distressing to AD patients.

PD is the second most common neurodegenerative disease, typically presenting as a progressive movement disorder with slowness, rigidity, gait difficulty, and tremor at rest. The pathological hallmarks of PD include the loss of dopamine (DA) neurons in the substantia nigra (SN) of the ventral midbrain and the presence of intracytoplasmic protein aggregates, termed Lewy bodies (LB), composed of the synaptic vesicle-associated protein αSynuclein (αSyn), ubiquitin, and other components. It is thought that the earliest pathological feature of PD is the loss of dopaminergic axonal processes that extend from the substantia nigra to the striatum, preceding the eventual loss of DA neuron cell bodies. PD pathology has been described broadly in the CNS and is not confined to midbrain DA neurons.

Epidemiological studies implicate both genetic and environmental factors in PD. However, molecular clues regarding the etiology of the disease were lacking until the identification of genes that underlie familial, inherited forms of Parkinsonism. Missense mutations and duplications in αSyn are associated with rare cases of autosomal dominant familial Parkinsonism. αSyn mutations lead to increased aggregation of the protein as well as altered vesicular trafficking and defective protein degradation through proteasome and lysosome pathways. The presence of αSyn aggregates in LB inclusions that typify sporadic PD support the notion that familial forms of Parkinsonism are informative with respect to the mechanism of sporadic PD. Mutations in Parkin, DJ-1, and Pink1 lead to autosomal recessive Parkinsonism and are associated with increased sensitivity to oxidative stress as well as mitochondrial dysfunction, further implicating these mechanisms in Parkinsonism. Autosomal dominant mutations in leucine-rich repeat kinase-2 (LRRK2, PARK8, dardarin, OMIM 609007) were described in a familial Parkinsonism syndrome that mimics the clinical and pathological features of the common, sporadic form of PD.

There is an ever increasing need for effective therapies to treat, prevent or slow the progression of neurodegenerative diseases. For example, blockers of glutamate release or antagonists of glutamate receptors, including NMDA receptors, have shown considerable importance as potential neuroprotective agents. To make significant progress towards treating neurodegenerative diseases such as AD, ALS and PD, it is important to identify molecular and genetic targets and use such targets to develop new therapeutic compounds and treatment strategies.

### SUMMARY

A method is disclosed for treating a neurodegenerative disease in a subject, the method comprising administering to the subject an effective amount of a compound of Formula I:

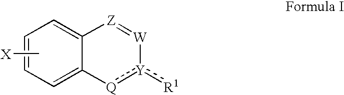

or a pharmaceutically acceptable base or acid addition salt, hydrate, stereoisomer, or mixture thereof, wherein X is one or more halogen radicals; Q is NH or N; W is CR², CHR², NR³, or CH═COH; Y is C or CH; Z is C═O, SO₂, COH, CHOH, or NHR⁴; R¹ is CO₂H or oxo (═O); R² is H, C(═O)—C₁-C₆ alkyl, C(═O)O—C₁-C₆ alkyl, or C(═O)—C₃-C₈ cycloalkyl; R³ is optionally substituted C₃-C₁₀ aryl; and R⁴ is

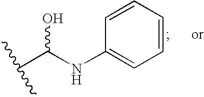

R² and R¹ combine with the carbons to which they are attached to form a 6 membered heterocycle that is optionally substituted at one or more of the heteroatoms with C₃-C₁₀ aryl, wherein the aryl may be substituted with one or more of C₁-C₆ alkyl or —O—C₁-C₆ alkyl, and the heteroatoms in the heterocyclic ring are one or more nitrogen atoms.

In one embodiment of the compound, X is one Cl radical at the 7 position of the fused benzene ring. In another embodiment, X is two Cl radicals at the 5 and 7 positions of the fused benzene ring. In another embodiment, Z is SO₂. In another embodiment, Z is C═O. In another embodiment, Z is COH. In another embodiment, Z is CHOH. In another embodiment, Z is NHR⁴, and R⁴ is

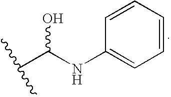

In another embodiment, Q is N, Y is C, and R¹ is CO₂H. In another embodiment, Q is NH, Y is C, and R¹ is oxo. In another embodiment, Q is NH, Y is CH, and R¹ is CO₂H. In another embodiment, W is CHR², and R² is C(═O)O—C₁-C₆ alkyl. In another embodiment, R² is C(═O)O-methyl. In another embodiment, W is CHR², and R² is C(═O)—C₃-C₈ cycloalkyl. In another embodiment, R² is C(═O)-cyclopropyl. In another embodiment, W is NR³. In another embodiment, R³ is benzyl. In another embodiment, R³ is benzyl substituted with a halogen. In another embodiment, W is NR³ and R³ is meta-bromo-benzyl.

A method for treating a neurodegenerative disease in a subject, the method comprising administering to the subject an effective amount of a compound of Formula II:

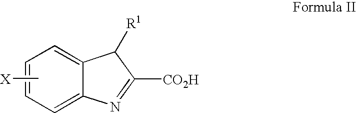

or a pharmaceutically acceptable base or acid addition salt, hydrate, stereoisomer, or mixture thereof, wherein X is one or more halogen radicals; R¹ is (CH₂)ₙ—CO₂H, or CH═CHC(═O)NHR²; and R² is C₃-C₁₀ aryl optionally substituted with one or more of C₁-C₆ alkyl, —O—C₁-C₆ alkyl, or halogen; and n is 0, 1, 2, 3, 4, 5, or 6.

In one embodiment, X is one Cl radical at the 7 position of the fused benzene ring. In one embodiment, X is two Cl radicals at the 5 and 7 positions of the fused benzene ring. In one embodiment, R¹ is CH₂CO₂H. In one embodiment, R¹ is (CH₂)₂CO₂H. In one embodiment, R¹ is CH═CHC(═O)NHR², and R² is phenyl.

In one embodiment, the compound is an antagonist of a NMDA receptor glycine site.

A method is provided for treating a neurodegenerative disease in a subject, the method comprising administering to the subject an effective amount of a compound selected from the group consisting of: ACEA 1012 (Licostinel), 5,7-dichlorokynurenic acid, L689,560, L701,252, L687,414, SC49648, MDL29,951, MDL105,519, GV150526 (Gavestinal) GV196771, RPR104,632, RPR118723, L695,902, ZD9379, 2-amino-5-phosphonopentanoate (AP-5), MK-801, L701,324, kynurenine, 1-aminocyclobutane carboxylic acid (ACBC), 1-aminocyclopentane-1-carboxylic acid (ACPC), AR-R15896AR, hydroquinone, and glutathione. In one embodiment, the treating comprises preventing the neurodegenerative disease, slowing the onset or progression of the neurodegenerative disease, alleviating one or more symptoms of the neurodegenerative disease, or any combination thereof. In one embodiment, the neurodegenerative disease comprises sporadic Parkinson's disease, autosomal recessive early-onset Parkinson's disease, Alzheimer's disease, stroke, amyotrophic lateral sclerosis, Binswanger's disease, Huntington's chorea, multiple sclerosis, myasthenia gravis or Pick's disease. In one embodiment, the neurodegenerative disease comprises a mutation in leucine-rich repeat kinase-2 (LRRK2).

A method is disclosed for treating a neurodegenerative disease, for example, Parkinson's Disease associated with a mutant of leucine-rich repeat kinase-2 (LRRK2), with NMDA antagonists. The methods include assessing accumulation of LC3-GFP labeled aggregates. The methods may be carried out on primary neurons in vitro in a cell culture, or in vivo in an animal. In one embodiment, the cell is a post-mitotic neuron. In another embodiment, the post-mitotic neuron is a cortical neuron, a dopamine neuron, or a sympathetic neuron.

A method is disclosed for treating impaired motor function associated with Parkinson's disease, anti-Parkinson's drug treatment, and/or dementia associated with Parkinson's disease. The invention is directed to the use of NMDA receptor antagonists for the treatment of impaired motor function.

A method of treating Parkinson's disease with an NMDA receptor antagonist is disclosed. In particular, the use of NMDA glycine site antagonists and related agents such as ACEA 1021, GV150526, GV196711, MDL 105,519, L-701324, L-687414, RPR 104632, ACPC, ZD9379, AR-R15896AR and RPR118723 are disclosed (See Table 1).

In one aspect, the invention provides a method for reducing LRRK2 mutant protein induced toxicity in a neuronal cell, the method comprising contacting the cell with a compound selected from the group comprising ACEA 1021, GV150526, GV196711, MDL 105,519, L-701324, L-687414, RPR 104632, ACPC, ZD9379, AR-R15896AR and RPR118723.

In another aspect, the invention provides a method for reducing the occurrence of LC3-GFP-labeled aggregates in cell, the method comprising contacting the cell with an NMDA receptor agonist selected from the group comprising ACEA 1021, GV150526, GV196711, MDL 105,519, L-701324, L-687414, RPR 104632, ACPC, ZD9379, AR-R15896AR and RPR118723.

In one embodiment, the neurodegenerative disease is selected from the group comprising: Alexander disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease (Spinocerebellar ataxia type 3), Multiple sclerosis, Multiple System Atrophy, Parkinson disease, Pelizaeus-Merzbacher Disease, Pick's disease, Primary lateral sclerosis, Refsum's disease, Sandhoff disease, Schilder's disease, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis, and any combination thereof. In another embodiment, the neurodevelopmental disorder comprises Angelman syndrome, Autism, Fetal Alcohol syndrome, Fragile X syndrome, Tourette's syndrome, Prader-Willi syndrome, Sex Chromosome Aneuploidy in Males and in Females, William's syndrome, Smith-Magenis syndrome, 22q Deletion, and any combination thereof.

The invention provides a method for determining whether a compound inhibits mutant leucine-rich repeat kinase-2 (LRRK2) protein activity, the method comprising (a) expressing in a primary neuronal cell a Parkinson's Disease-associated LRRK2 mutant protein, wherein expression of the mutant results in accumulation of axonal spheroid inclusions in the cell that stain positive for Tau protein; (b) contacting the neuronal cell with a compound; and (c) determining whether accumulation of axonal spheroid inclusions in the neuronal cell is reduced compared to accumulation of axonal spheroid inclusions in a neuronal cell expressing the LRRK2 mutant in the absence of the compound, wherein determination of a reduction in (c) indicates that the compound inhibits the LRRK2 mutant protein activity.

The invention also provides a method for determining whether a compound inhibits mutant leucine-rich repeat kinase-2 (LRRK2) protein activity, the method comprising (a) expressing in a primary neuronal cell a Parkinson's Disease-associated LRRK2 mutant protein, wherein expression of the mutant results in decreased axonal length; (b) contacting the neuronal cell with a compound; and (c) determining whether axonal length in the neuronal cell is increased compared to axonal length in a neuronal cell expressing the LRRK2 mutant in the absence of the compound, wherein determination of an increase in (c) indicates that the compound inhibits the LRRK2 mutant protein activity.

The invention further provides a method for determining whether a compound inhibits mutant leucine-rich repeat kinase-2 (LRRK2) protein activity, the method comprising (a) expressing in a neuronal cell a Parkinson's Disease-associated LRRK2 mutant, wherein expression of the mutant results in decreased axonal branching; (b) contacting the neuronal cell with a compound; and (c) determining whether axonal branching in the neuronal cell is increased compared to axonal branching in a neuronal cell expressing the LRRK2 mutant in the absence of the compound, wherein determination of an increase in (c) indicates that the compound inhibits the LRRK2 mutant protein activity.

In other embodiments, the primary neuronal cell comprises a nucleic acid vector encoding a Parkinson's Disease-related LRRK2 mutant. In one embodiment, the vector is a lentiviral vector, an adeno-associated virus-2 (AAV-2) vector, an adenoviral vector, a retroviral vector, a polio viral vector, a murine Maloney-based viral vector, an alpha viral vector, a pox viral vector, a herpes viral vector, a vaccinia viral vector, a baculoviral vector, a parvoviral vector, or any combination thereof. In another embodiment, the vector comprises a nucleic acid sequence encoding a fragment of a LRRK2 protein. In another embodiment, the vector comprises a nucleic acid sequence encoding a LRRK2 kinase domain.

In other embodiments, the method further comprises expressing a fluorescent protein in the primary neuronal cell.

In other embodiments, the LRRK2 mutant protein consists essentially of a LRRK2 kinase domain, wherein the kinase domain comprises one or more Parkinson's Disease-related LRRK2 mutations. In other embodiments, the LRRK2 mutant protein comprises a G2019S mutation, an I2020T mutation, or both. The locations of the LRRK2 mutations are based on the amino acid sequence (SEQ ID NO:1) translated from the mRNA sequence of human LRRK2 (SEQ ID NO:2) (GenBank Accession No. AY_{—}792551).

In additional embodiments, the primary neuronal cell is in a cell culture. In further embodiments, the primary neuronal cell is in vivo in an animal. In other embodiments, the primary neuronal cell is a post-mitotic neuron. In another embodiment, the post-mitotic neuron is a cortical neuron, a dopamine neuron, or a sympathetic neuron.

In one embodiment, the determining comprises detecting fluorescence. In another embodiment, the determining comprises computer-assisted quantification of axonal length. In another embodiment, the determining comprises computer-assisted quantification of axonal branching.

In another embodiment, the compound inhibits glycogen synthase kinase 3 beta (GSK3β). In another embodiment, compound activates an AKT (protein kinase B) protein, downstream components in an AKT pathway, or both.

In an additional embodiment, the compound comprises a peptide fragment of a LRRK2 protein. In another embodiment, the peptide comprises a LRRK2 kinase domain. In another embodiment, the peptide consists essentially of a LRRK2 kinase domain. In another embodiment, the peptide consists of a LRRK2 kinase domain.

In one embodiment, the compound comprises a nucleic acid, or a polypeptide expressed therefrom, capable of inhibiting expression of a LRRK2 protein. In another embodiment, the compound comprises a nucleic acid comprising RNA, antisense RNA, small interfering RNA (siRNA), double stranded RNA (dsRNA), short hairpin RNA (shRNA), cDNA, DNA, or any combination thereof. An example of an shRNA construct provided by the invention targets bases 4789-4809 of the rodent LRRK2 gene with GenBank Accession No. NM_{—}025730 (SEQ ID NO:3). The invention also provides for nucleic acid inhibitors that target the human LRRK2 gene (for example, see Accession No. NM_{—}198578), or fragments thereof.

In one embodiment, the compound is an anti-oxidant. In another embodiment, the compound is a retinoid. In yet another embodiment, the compound is a N-methyl-D-aspartic acid (NMDA) receptor antagonist.

In one embodiment, the method is carried out in a multi-well plate. In another embodiment, the method is carried out in a high-throughput manner. In another embodiment, the method is carried out for more than one hundred compounds in a high-throughput manner.

A viral vector is provided that comprises a nucleic acid encoding a LRRK2 kinase domain, or a fragment thereof, wherein the kinase domain comprises one or more Parkinson's Disease-associated LRRK2 mutations.

A method is provided for inhibiting activity of a Parkinson's disease-associated LRRK2 mutant protein in a neuronal cell, the method comprising contacting the cell with an NMDA receptor antagonist. One embodiment provides a method for inhibiting activity of a Parkinson's disease-associated LRRK2 mutant protein in a neuronal cell, the method comprising contacting the cell with an antioxidant. Another embodiment provides a method for inhibiting activity of a Parkinson's disease-associated LRRK2 mutant protein in a neuronal cell, the method comprising contacting the cell with a compound selected from the group consisting of: 2-amino-5-phosphonopentanoate (AP-5), MK-801, L-701,324, kynurenine, 1-aminocyclobutane carboxylic acid (ACBC), hydroquinone, and glutathione.

A method is provided for increasing axonal length, axonal branching, or both in a neuronal cell, the method comprising contacting the cell with an NMDA receptor antagonist. A method is provided for increasing axonal length, axonal branching, or both in a neuronal cell, the method comprising contacting the cell with an antioxidant. A method is provided for increasing axonal length, axonal branching, or both in a neuronal cell, the method comprising contacting a neuronal cell with a compound selected from the group consisting of: 2-amino-5-phosphonopentanoate (AP-5), MK-801, L-701,324, kynurenine, 1-aminocyclobutane carboxylic acid (ACBC), hydroquinone, and glutathione.

In one embodiment, the NMDA receptor antagonist is 2-amino-5-phosphonopentanoate (AP-5), MK-801, L-701,324, kynurenine, 1-aminocyclobutane carboxylic acid (ACBC) or hydroquinone. In another embodiment, the antioxidant is glutathione or hydroquinone.

A method is provided for treating a neurodegenerative disease in a subject, the method comprising administering to the subject an effective amount of a compound selected from the group consisting of: 2-amino-5-phosphonopentanoate (AP-5), MK-801, L-701,324, kynurenine, 1-aminocyclobutane carboxylic acid (ACBC), hydroquinone, and glutathione.

### BRIEF DESCRIPTION OF THE FIGURES

FIGS. 1A-1I. Expression, kinase activity, and localization of LRRK2. FIG. 1A, Schematic of the primary structure of LRRK2 including the leucine rich repeat (LRR), Roc, COR, protein kinase (PK), and WD-repeat domains. Clinical mutations (Y1699C, G2019S, I2020T) and the putative dominant negative allele (K1906M) are shown. FIG. 1B, Upper panel displays an autoradiograph of the in vitro kinase assay with myosin light chain substrate. Lower panel shows a Western blot for the V5-epitope tag, demonstrating equal expression of the LRRK2 alleles and deletions. V5 epitope tagged wild-type or mutant alleles of LRRK2 were overexpressed in 293T cells. Cell lysates were immunoprecipitated with an antibody to the V5 epitope-tag, and in vitro protein kinase assays performed with myosin light chain as a substrate. FIG. 1C, Knockdown of LRRK2 with transfection of shRNA or vector control in transfected primary rat P1 cortical cultures. Immunohistochemical analysis was performed with a polyclonal antibody to LRRK2. LRRK2 protein is found throughout the cytoplasm and neurites, and is enriched at the cell cortex and at membrane structures. LRRK2 shRNA vector transfected cells display reduced LRRK2 staining relative to untransfected cells in the same culture. Insets are magnified (2.5×) views of the transfected soma. FIG. 1D, Quantification of knockdown efficiency was performed as in FIG. 1C using NIH Image software (P<0.005, n=3 for each group). FIG. 1E, Localization of wild-type or G2019S mutant alleles of LRRK2 in transfected primary rat P1 cortical cultures. Immunohistochemical analysis was performed with an antibody to the polyhistidine (His) epitope-tag. Immunoreactivity is detected throughout the soma and neuronal processes (see insets; 3× magnified), as well as in inclusions (arrow). FIG. 1F, Overexpression of V5 epitope-tagged wild-type or mutant alleles of LRRK2 in COS-7 cells. Western blots were performed with antibodies to the V5 epitope tag, to LRRK2 protein, or to GSK3β (as a loading control). FIG. 1G, Cell lysates (as in FIG. 1C) were immunoprecipitated with an antibody to the V5 epitope-tag, and protein kinase assays performed with myelin basic protein (MBP) as a substrate. Upper panel shows an autoradiograph of the in vitro kinase assay; lower panel is a Western blot for the V5 epitope-tag to demonstrate equal expression of the LRRK2 alleles and deletion forms. FIG. 1H, Knockdown of LRRK2 with transfection of shRNA vectors in C6 rat glioma cells quantified by (i) Western blotting of cell lysates with an antibody to LRRK2 or (ii) real-time quantitative RT-PCR analysis of mRNA. FIG. 1I, Localization of wild-type or G2019S mutant alleles of LRRK2 in transfected primary rat P1 cortical cultures. Immunohistochemical analysis was performed with a polyclonal antibody to LRRK2. Cells are cotransfected with GFP marker plasmid, and GFP-positive cells display increased (approximately 2-fold) expression of LRRK2, in agreement with Western blot analysis in FIG. 1F.

FIGS. 2A-2C. LRRK2 regulates neuronal process morphology in P1 primary rat cortical cultures. FIG. 2A, Camera lucida drawings of LRRK2-transfected cortical neurons. Wild type, G2019S, I2020T, R1441G, Y1699C, or K1906M LRRK2 alleles were transfected into cortical cultures at day 7 in vitro (7 DIV). At 2 weeks after transfection, cultures were imaged by confocal microscopy and process length was quantified by an observer blind to the identity of the allele. Process complexity and length was reduced significantly in the G2019S (n=55) and I2020T (n=20) cultures relative to control vector (n=10) or wild type (WT) LRRK2 (n=55), whereas the putative dominant negative allele, K1906M (n=20), leads to an increase in process length. A double mutant allele that harbors both the K1906M and G2019S mutations mimics the dominant negative phenotype. Knockdown of LRRK2 with an shRNA vector (n=10) leads to increased process length relative to vector control (n=10). FIG. 2B, Quantification of soma diameter, total process length, longest process length, and the number of branch points off of the longest process. *, p<0.05. FIG. 2C, Transfected cortical cultures, as above, were immunostained with a mouse monoclonal antibody to Tau (Tau-1), which marks axonal processes primarily, or with a rabbit polyclonal antibody to MAP2, which marks dendritic processes. Arrows mark the axonal processes, asterisks denote the soma and surrounding dendritic processes.

FIGS. 3A-3G. Time course analysis of LRRK2 process phenotype. FIG. 3A, Primary rat cortical P1 cultures were transfected with LRRK2 alleles at 7 DIV, as indicated, and process length and branching were quantified over time subsequently. Overexpression of mutant, but not WT, LRRK2 leads to a progressive decrease in the number of branch points of the longest process, and a subsequent decrease in process length, over a 15 day period. In contrast, LRRK2 knockdown by shRNA leads to a significant progressive increase in process length (n=15 per group). FIG. 3B, Overexpression of mutant LRRK2 alleles leads to a significant increase in apoptotic cell death, as visualized by propidium iodide (PI) staining and activated Caspase-3 immunostaining, and a corresponding reduction in survival, as determined by nuclear morphology. FIG. 3C, Quantification of cell survival; mutant LRRK2 alleles lead to significantly less survival that WT LRRK2 or control vector (*, P<0.005). n=15 for each group. FIG. 3D, Rescue of the LRRK2 shRNA knockdown requires only the kinase domain of the protein. Primary neuronal cultures were transfected with full-length, GTPase/Cor/kinase (deleted in amino- and carboxy-terminal domains), or the kinase domain alone. n=15 for each group. FIG. 3E, Deletion analyses of LRRK2 G2019S and wild-type (WT) alleles demonstrate a role for the kinase domain in the neuron process length phenotype. The G2019S kinase domain alone is sufficient to reduce process length, and leads to a more dramatic phenotype than the full-length protein or a truncated protein that harbors GTPase, COR, and kinase domains. The WT GTPase domain alone displays no activity. n=10 for each group. FIG. 3F, Primary rat P1 midbrain cultures were transduced at 7 DIV with adenoassociated virus (AAV) or lentivirus (LV) viral vectors that harbor eGFP along with WT or G2019S mutant LRRK2 kinase domains or LRRK2 shRNA and analyzed by confocal microscopy for eGFP (green) and immunohistochemistry for tyrosine hydroxylase (TH; red) to identify dopamine neurons. ShRNA mediated knockdown of LRRK2 but not control vector (15 cells each) led to a significant increase in longest process length. In contrast, overexpression of the G2019S LRRK2 mutant allele led to a significant reduction in process length (9 cells), in comparison with control vector (10 cells) or WT LRRK2 (3 cells). FIG. 3G, Quantification of dopamine neuron process length as in FIG. 3F.

FIGS. 4A-4C. Tau-positive spheroidal inclusions in neurons that overexpress mutant G2019S LRRK2 mutations. FIG. 4A, Confocal microscopy for GFP marker or immunohistochemistry for the His-tag epitope on LRRK2 G2019S demonstrate the presence of spheroid inclusions within processes of all transfected cells (identical results were obtained with the 12020T mutation). LRRK2 protein does not appear to be enriched in the inclusions relative to the GFP marker. The inclusions additionally stain with antibodies for Tau phosphorylated at serine 202 (P-Tau) and total Tau, but not with an antibody for α-Synuclein. Arrows point to inclusions. FIG. 4B, Quantification of aggregates as in FIG. 4A. Inclusions are infrequent with WT LRRK2 overexpression (11.1%) and vector alone (3.5%). Aggregates are significantly more abundant in cells expressing mutant LRRK2 than in cells expressing WT LRRK2 or vector alone (P<0.006). WT LRRK2 expression does lead to an increase in aggregate formation relative to vector alone (P<0.02). N>20 for each group. FIG. 4C, Time course analysis of inclusion formation (quantified as in FIG. 4B and neuronal survival (quantified as in FIG. 3). Aggregation is observed as early as 6 DIV after transfection, whereas decreased survival is observed after 12 days. N=15 for each group.

FIGS. 5A-5E. Overexpression of LRRK2 G2019S kinase domain leads to Tau-positive inclusions, increased apoptosis, and altered process morphology in rat nigral dopamine neurons. FIG. 5A, Adult rats were transduced unilaterally into the substantia nigra with AAV2 vectors harboring the G2019S or wild-type LRRK2 kinase domain, or empty vector, along with AAV2-GFP. Pathological examination was performed at 1 month. G2019S or wild-type LRRK2 kinase domain transduction leads to the appearance of axonal inclusions enriched in Tau, phospho-Tau (at serine 404) and VMAT2, as well as structural axonal defects (see FIG. 5D). Scale bar, 20 mm. FIG. 5B, Similar levels of transduction (approximately 85%; see FIG. 5E) were observed in the substantia nigra for all vectors; scale bar—100 μm. Activated caspase-3 staining in the nucleus of TH positive infected neurons (indicated by white arrows) was seen to increase in G2019S LRRK2 kinase domain transduced cells; Scale bar—100 μm in upper panels, 10 μm in lower panels. FIG. 5C, LRRK2 G2019S or wild-type kinase domain transduction leads to a significant accumulation of inclusions relative to control vector alone. Inclusions in the striatum (greater than 5 μm in diameter) were quantified. LRRK2 G2019S kinase domain transduction also resulted in a significant increase in activated caspase-3 staining localized in the nucleus. N=8 animals in each group. P<0.05; **. FIGS. 5D and 5E, Striatal dopaminergic (TH positive) axonal projections appear reduced in complexity when transduced with the G2019S kinase domain as compared with GFP infected axons. Transduction with the G2019S kinase domain also results in inclusions visible on the axons as seen in FIGS. 5A-5C. Scale bar—30 μm.

FIGS. 6A-6C. LRRK2 regulates neuronal process morphology in the developing neocortex. FIG. 6A, To examine the role of LRRK2 on brain development, cDNA for WT or mutant alleles of LRRK2, or LRRK2 shRNA, were introduced along with a GFP reporter into neural progenitor cells in E16 rat neocortex by in utero electroporation or lentiviral transduction. GFP positive cells were examined by confocal microscopy 4 days later. Electroporation of G2019S or I2020T alleles of LRRK2 led to a significant reduction in longest process length relative to WT LRRK2 or control vector plasmid. Branch point number was reduced with overexpression of either WT or mutant LRRK2 alleles relative to the vector control. In contrast, knockdown of LRRK2 by shRNA lentiviral transduction resulted in a significant increase in process branching relative to lentiviral vector control (which is not significantly different from the plasmid vector control). LRRK2 knockdown resulted in a small increase in the length of the longest process, but the effect was not significant (P=0.104). Branch points are indicated by white arrows. Scale bar, 20 μm. The neuronal identity of GFP positive cells is confirmed by immunostaining with an antibody for TuJI, shown in the lower right panel in FIG. 6A. The position of the GFP-positive cell body is indicated by a white trace. Scale bar—15 μm. N>25 for each group. FIG. 6B, Quantification of neuron process length and branching as in (A); *, P<0.05; **, P<0.005. FIG. 6C, Cells transduced with LRRK2 G2019S and GFP display comparable migration patterns to cells transduced with GFP alone. Scale bar—70 μm. FIGS. 7A-7I. Evidence for early lysosomal abnormalities in G2019S LRRK2 mutant allele expressing neurons. FIG. 7A, Ultrastructural analysis of primary cortical neurons expressing LRRK2 G2019S mutant allele or control vector by electron microscopy. LRRK2 expressing cells harbor abundant electrondense structures that are suggestive of swollen lysozomes (arrow), as well as multivessicular bodies (asterisk) and distended mitochondria associated with vacuoles (arrowhead). At highest magnification, membranes appear to surround the inclusions. FIG. 7B, Primary rat cortical cultures overexpressing the G2019S LRRK2 allele were immunostained with an antibody to LAMP 1, a membrane marker for acidic organelles including lysosomes and late endosomes. Staining is apparent at neurite inclusions (arrow) and colocalizes with LRRK2. FIG. 7C, Dopaminergic axons in the striatum transduced with the G2019S LRRK2 kinase domain stain were immunstained with antibodies for LC3, an autophagic marker, and Cathepsin D, a late endosome and lysosomal marker. Both LC3 and Cathepsin D marked TH-positive inclusions (arrows) in the G2019S transduced axons, but these are absent from control transduced processes (see FIG. 5). FIG. 7D, Primary rat P1 cultures transfected with G2019S or wild type LRRK2 alleles, LRRK2 shRNA, or control vector, were stained at 5 or 12 days after transfection with Lysotracker, a dye that stains acidic organelles such as lysosomes and late endosomes. At left, confocal micrographs are shown at the 5-day time point, demonstrating abnormal accumulation at inclusions (arrow) in the context of the G2019S mutant allele. This is further increased at the 12 day time point (right panel). FIG. 7E, Mitotracker dye analysis of primary neuronal cultures that overexpress either wild-type or G2019S mutant LRRK2; FIG. 7F, Mutant LRRK2 appears to interact with the AKT signaling pathway. Overexpression of a constitutively active form of AKT1 (c.a.-AKT1) dramatically increases process length and branching, particularly with respect to the longest process. Co-expression of G2019S mutant, but not WT, LRRK2 completely suppresses this phenotype. N=10 per group. *, P<0.05. FIG. 7G, c.a.-AKT1 fails to rescue the inclusion phenotype of the G2019S expressing neurons, and thus this phenotype is separable from survival. Arrows point to inclusions. FIGS. 7H and 7I, Analysis of vesicular endocytosis using FM4-64 in primary neuronal cultures transduced with either wild-type or G2019S mutant LRRK2. FM4-64 uptake puncta (arrows) appeared unaltered in the context of G2019S mutant allele expression as a function of unit length of process. Overall process length was reduced as described above, and therefore the number of uptake puncta per neuron was reduced. FM4-64 (10 μM) was loaded with hyperkalemic solution containing (in mM): NaCl (34); KCl (90); HEPES (20); CaCl₂ (2); MgCl₂ (2); glucose (30); pH 7.3 for 45 seconds and then washed with normal bath solution containing (in mM): NaCl (119); KCl (5); HEPES (20); CaCl₂ (2); MgCl₂ (2); glucose (30); pH 7.3 for 10 min. The cells were viewed with Zeiss LSM 510 confocal microscope. GFP and FM 4-64 were both excited by light at a wavelength of 488 nm, and viewed with emission filter ranges of 505-550 nm and 700-850 nm, respectively.

FIGS. 8A-8B. Antioxidants and NMDA receptor blockade suppress the G2019S LRRK2-associated toxicity, but not inclusion formation. FIG. 8A, Glutathione, hydroquinone, kynurenine, 1-aminocyclobutane carboxylic acid, and flavanone (10 μM) all significantly suppress the neurite toxicity of the G2019S LRRK2 allele, but do not alter process length in wild-type ovexpressing cells. Suppression is presented as a ratio of the G2019S longest process length over the wild-type longest process length in the context of each of the drugs, as a percentage of vehicle (0,1% DMSO). On the left, examples of neurons treated with glutathione versus vehicle show that the G2019S toxicity is suppressed, but that inclusions (arrows) remain in these cells. N=25 for each group. FIG. 8B, Constitutively active (c.a.) AKT1 or dominant negative (d.n.) GSK3β rescue the decreased neuronal survival associated with G2019S expressing neurons. Similarly, these constructs rescue the neurite length phenotype, but not the inclusion formation, found in G2019S expressing cells (see also FIGS. 7E-7G). N=10 for each group.

FIG. 9. Chemical structures of kynurenine, 1-aminocyclobutane carboxylic acid (ACBC), hydroquinone and glutathione.

FIGS. 10A-10C. Analysis of LRRK2-associated toxicity (FIG. 1A), inclusion formation (FIG. 10B) and cell survival (FIG. 10C) in neurites treated with 20 μM each of the compounds indicated on the y-axis of FIG. 10B.

FIG. 11. Structure of ACEA 1021. IUPAC designation 6,7-dichloro-5-nitro-1,4-dihydroquinoxaline-2,3-dione.

FIG. 12. Structure of GV150526. IUPAC designation 4,6-dichloro-3-[(E)-3-(cyclohexylamino)-3-oxoprop-1-enyl]-1H-indole-2-carboxylic acid.

FIG. 13. Structure of GV196771. IUPAC designation E-4,6-dichloro-3-(2-oxo-1-phenyl-pyrrolidin-3-glydenemethyl)-1H-indole-2-carboxylic acid).

FIG. 14. Structure of MDL 1 05,519. IUPAC designation (E)-3-(2-phenyl-2-carboxyethenyl)-4,6-dichloro-1H-indole-2-carboxylic acid.

FIG. 15. Structure of L-701324. IUPAC designation 7-chloro-2-hydroxy-3-[3-(phenoxy)phenyl]-1H-quinolin-4-one.

FIG. 16. Structure of L-687414. IUPAC designation (3S,4S)-3-amino-1-hydroxy-4-methylpyrrolidin-2-one.

FIG. 17. Structure of RPR 104632. IUPAC designation 2H-1,2,4-benzothiadiazine-1-dioxide-3-carboxylic acid.

FIG. 18. Structure of ACPC. IUPAC designation 1-aminocyclopropane-1-carboxylic acid.

FIG. 19. Structure of ZD9379. IUPAC designation 7-chloro-2,3-dihydro-2-(4-methoxy-2-methylphenyl)pyridazino[4,5b]quinoline-1,4,10(5H)-trione

FIG. 20. Structure of AR-R15896AR. IUPAC designation [RS]-alpha-phenyl-2-pyridine-ethanamine.

FIG. 21. Structure of RPR 118723. IUPAC designation 8-chloro-5-methyl-2,3-dioxo-1,4-dihydro-5H-indeno[1,2-b]pyrazin-5-yl)acetic acid.

FIG. 22. Non-limiting examples of compounds that can be used in the methods described herein.

### DETAILED DESCRIPTION

The issued patents, applications, and other publications that are cited herein are hereby incorporated by reference to the same extent as if each was specifically and individually indicated to be incorporated by reference.

As disclosed herein, antagonists of the N-methyl-D-aspartate (NMDA) receptor glycine site can be used to treat neurodegenerative disease. The disclosure provides methods for treating neurodegenerative disease and compounds that can be used in the disclosed methods. Also provided are compound screening methods based on Parkinson's disease-associated mutations in leucine-rich repeat kinase-2 (LRRK2).

N-methyl-D-aspartate (NMDA) glutamate receptors have several important functions in the motor circuits of the basal ganglia, and represent an important target new compounds in the prevention or treatment of Parkinson's disease (PD). NMDA receptor ligand-gated ion channels are composed of multiple subunits and have agonist and co-agonist binding sites. NMDA receptors in the striatum function in dopaminic-glutamate interactions. The abundance, structure, and function of striatal receptors are altered by the dopamine depletion and further modified by the pharmacological treatments used in PD. In animal models, NMDA receptor antagonists are effective anti-parkinsonian agents and can reduce the complications of chronic dopaminergic therapy. Use of these agents in humans has been limited because of the adverse effects associated with nonselective blockade of NMDA receptor function, but the development of more potent and selective pharmaceuticals holds the promise of in important new therapeutic approach for PD.

Autophagy is a degradative mechanism involved in the recycling and turnover of cytoplasmic constituents from eukaryotic cells. This phenomenon of autophagy has been observed in neurons from patients with PD, suggesting a functional role for autophagy in neuronal cell death. Autophagic cell death involves accumulation of autophagic vacuoles (AVs) in the cytoplasm of dying cells as well as mitochondria dilation and enlargement of the endoplasmic reticulum and the Golgi apparatus. Autophagic cell death has been described during the normal nervous system development and could be a consequence of a pathological process such as those associated with neurodegenerative diseases. The formation of AV can be measured by the accumulation of the autophagosome marker LC3 to AV in discreet foci.

Leucine-rich repeat kinase-2 (LRRK2, PARK8, dardarin) (Online Mendelian Inheritance in Man (OMIM) No. 609007) (GenBank Accession No: NM_{—}025730) encodes a multidomain protein that includes a Rho/Ras-like GTPase domain (termed Roc, for Ras in complex proteins), a protein kinase domain related to the mixed lineage kinase (MLK) family (Manning et al., 2002), as well as WD40-repeat and LRR domains. An additional domain C-terminal to the GTPase domain, termed COR (for carboxy-terminal of Ras), is of unknown function but typifies the ROCO family of related proteins which harbor both GTPase and protein kinase-like domains (Bosgraaf and Van Haastert, 2003). PD-associated mutations in LRRK2 fall throughout all of the identified structural segments. The G2019S and 12020T mutations are both predicted to alter a highly conserved region of the kinase domain termed the ‘activation loop’, based on structural homology to other protein kinases (Davies et al., 2002), and mutations within this segment lead to disinhibited kinase activity in vitro (Gloeckner et al., 2005; West et al., 2005).

LRRK2 mutations appear to be relatively common genetic determinants of PD susceptibility: a single missense allele of LRRK2, G2019S, may be associated with 1-2% of apparently ‘sporadic’ PD cases (Di Fonzo et al., 2005; Gilks et al., 2005; Nichols et al., 2005), and, with over 10% of PD cases in specific populations such as Ashkenazi Jews and North African Arabs (Lesage et al., 2006; Ozelius et al., 2006). No prior mutation has been associated with a significant proportion of PD patients. Therefore, it is of great clinical interest to understand LRRK2 function and mechanism of action, as they may play important roles in the pathogenesis of PD and possibly other neurodegenerative diseases.

Pathological examination of patients with LRRK2 mutations has revealed dopamine (DA) neuron degeneration in the substantia nigra (SN) of the ventral midbrain, as expected, but also heterogeneity regarding other pathological features: some cases harbor αSynuclein (αSyn)-positive Lewy body (LB) intracytoplasmic aggregates typical of sporadic PD and other synucleinopathies; whereas other cases either lack LB aggregates, display widespread LB pathology in the cerebral cortex, or harbor Tau-positive axonal inclusions (Wszolek et al., 2004).

Prior studies of PD-associated genes have focused on mechanisms of oxidative stress and mitochondrial dysfunction. The present invention provides that an early consequence of LRRK2 mutations is altered regulation of axonal process maintenance and morphology.

The disclosed subject matter provides that expression of a PD-associated LRRK2 mutant in a primary neuronal cell, such as a cortical neuron or midbrain dopamine neuron, results in accumulation of axonal spheroid inclusions that stain positive for Tau protein, decreased axonal length, decreased axonal branching, reduced survival of the neuronal cell, or any combination thereof.

LRRK2 clinical mutations, including G2019S, lead to a reduction in the length and complexity of cortical neuron processes, and this is associated with increased kinase activity. In contrast, suppressing LRRK2 activity, for example by using a dominant negative allele or RNA interference, leads to an increase in neuron process length and complexity. It is a further discovery of the invention that LRRK2 is associated with the regulation of neuronal process morphology, and that the LRRK2 kinase domain is necessary and sufficient for this activity. Additionally, it is a discovery of the invention that the LRRK2 kinase domain is necessary and sufficient for the regulation of neuronal process morphology. The invention provides cellular and animal models of LRRK2 mutant-associated pathology and compound screening methods. The invention further provides methods for analyzing LRRK2 activity in vivo using in utero cortical neuron electroporation in rat embryos.

The invention provides that clinical mutations in the kinase domain ‘activation loop’ of LRRK2 lead to disinhibited kinase activity. Using a structure/function approach, the invention identifies inhibitory domains of the protein that negatively regulate kinase activity.

In primates, dopamine neurons accumulate pigmented lysosome-related organelles that contain neuromelanin and metals (Zecca et al., 2003), proposed to play both pathological and protective roles in PD. The invention also provides that an early feature of cells that overexpress clinical mutants of LRRK2 is the accumulation of abnormal late endosome lysosomes. Endogenous LRRK2 was found to be associated with late endosomal and lysosomal membranes. Expression of mutant LRRK2 alleles induces large intracellular inclusions over time, and these harbor LRRK2 protein as well as lysosomal and endosomal markers. These inclusions also contain phosphorylated Tau protein, consistent with the pathology observed in patient autopsy material. Endogenous LRRK2 appears concentrated around endosome and lysosome compartments, and these compartments are abnormally accumulated in neurons that express mutant LRRK2. Endosome and lysosome trafficking play dominant roles in process outgrowth in neurons, and mutations in other genes that regulate these compartments also alter neurite outgrowth. Thus, these findings present evidence for a cellular mechanism by which LRRK2 regulates neuronal process outgrowth.

### Compounds

The methods provided can be used in a chemical genetic approach for screening compounds (see Example 1). Using the disclosed methods, NMDA receptor antagonists or partial agonists, (for example, 2-amino-5-phosphonopentanoate (AP-5), MK-801, L-701,324, kynurenine, 1-aminocyclobutane carboxylic acid (ACBC) and hydroquinone) and antioxidants (for example, glutathione, hydroquinone and flavanone) were found to effectively block LRRK2 toxicity, implicating oxidative stress and glutamate excitotoxicity in LRRK2 toxicity. Other examples of compounds that can be used in the disclosed methods include NMDA glycine site antagonists such as ACEA 1021 (Licostinel), GV150526 (Gavestinal), GV196711, MDL 105,519, L-701,324, L-687414, RPR 104632, ACPC, ZD9379, and RPR118723 (see Example 2). Compounds of Formula I or Formula II as described herein may be used in the disclosed methods.

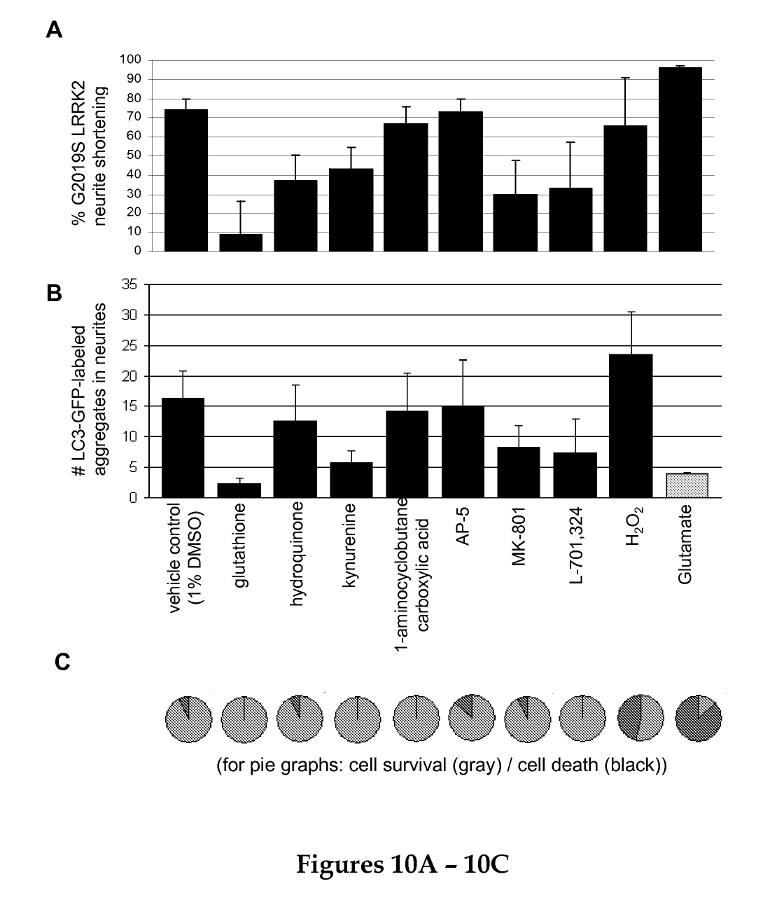

Kynurenine is a NMDA glycine site antagonist. Kynurenine may be used in the disclosed methods. Non-limiting examples of kynurenine structural analogs which bind to the same site as kynurenine (i.e., the NMDA glycine site) include 5,7-dichlorokynurenic acid, L689,560, L701,252, L701,324 (see FIGS. 10A-10C), SC49648, MDL29,951, GV150526A, RPR104,632, L695,902, ZD9379, and 1-aminocyclobutane carboxylic acid. Examples of kynurenine analogs are described in Stone and Darlington, “Endogenous Kynurenines as Targets for Drug Discovery and Development” Nature Reviews 1:609-620 (2002), which is herein incorporated by reference.

The invention provides compounds of Formula I and Formula II. Such compounds may be used in the disclosed methods.

One embodiment provides compounds of Formula I:

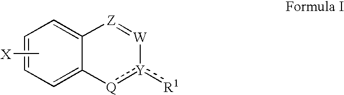

wherein

X is one or more halogen radicals;

Q is NH or N;

W is CR², CHR², NR³, or CH═COH;

Y is C or CH;

Z is C═O, SO₂, COH, CHOH, or NHR⁴;

R¹ is CO₂H or oxo (═O);

R² is H, C(═O)—C₁-C₆ alkyl, C(═O)O—C₁-C₆ alkyl, or C(═O)—C₃-C₈ cycloalkyl;

R³ is optionally substituted C₃-C₁₀ aryl; and

R⁴ is

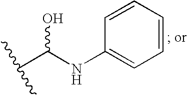

R² and R¹ combine with the carbons to which they are attached to form a 6 membered heterocycle that is optionally substituted at one or more of the heteroatoms with C₃-C₁₀ aryl, wherein the aryl may be substituted with one or more of C₁-C₆ alkyl or —O—C₁-C₆ alkyl, and the heteroatoms in the heterocyclic ring are one or more nitrogen atoms.

In one embodiment, X is one Cl radical at the 7 position of the fused benzene ring.

In another embodiment, X is two Cl radicals at the 5 and 7 positions of the fused benzene ring.

In one embodiment, Z is SO₂.

In another embodiment, Z is C═O.

In another embodiment, Z is COH.

In another embodiment, Z is CHOH.

In another embodiment, Z is NHR⁴, and R⁴ is

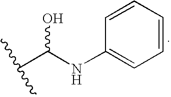

In one embodiment, Q is N, Y is C, and R¹ is CO₂H.

In another embodiment, Q is NH, Y is C, and R¹ is oxo.

In another embodiment, Q is NH, Y is CH, and R¹ is CO₂H.

In one embodiment, W is CHR², and R² is C(═O)O—C₁-C₆ alkyl.

In one embodiment, R² is C(═O)O-methyl.

In another embodiment, W is CHR², and R² is C(═O)—C₃-C₈ cycloalkyl.

In one embodiment, R² is C(═O)-cyclopropyl.

In another embodiment, W is NR³.

In one embodiment, R³ is benzyl

In another embodiment R³ is benzyl substituted with a halogen.

In another embodiment, W is NR³ and R³ is meta-bromo-benzyl.

In another embodiment, a compound of Formula I is not a naturally-occurring compound. In another embodiment, a compound of Formula I is not Compound 1, 2, 3, 4, 8, 9, 10 or 11 (shown below).

Another embodiment provides a compound of Formula II:

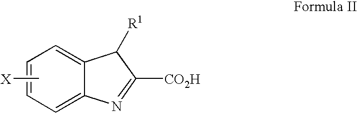

wherein

X is one or more halogen radicals;

R¹ is (CH₂)ₙ—CO₂H, or CH═CHC(═O)NHR²; and

R² is C₃-C₁₀ aryl optionally substituted with one or more of C₁-C₆ alkyl, —O—C₁-C₆ alkyl, or halogen; and

n is 0, 1, 2, 3, 4, 5, or 6.

In one embodiment, X is one Cl radical at the 7 position of the fused benzene ring.

In another embodiment, X is two Cl radicals at the 5 and 7 positions of the fused benzene ring.

In one embodiment, R¹ is CH₂CO₂H.

In another embodiment, R¹ is (CH₂)₂CO₂H.

In yet another embodiment, R¹ is CH═CHC(═O)NHR², and R² is phenyl.

In another embodiment, a compound of Formula II is not a naturally-occurring compound. In another embodiment, a compound of Formula II is not Compound 5, 6, or 7 (shown below).

Non-limiting examples of compounds of Formula I include the following:

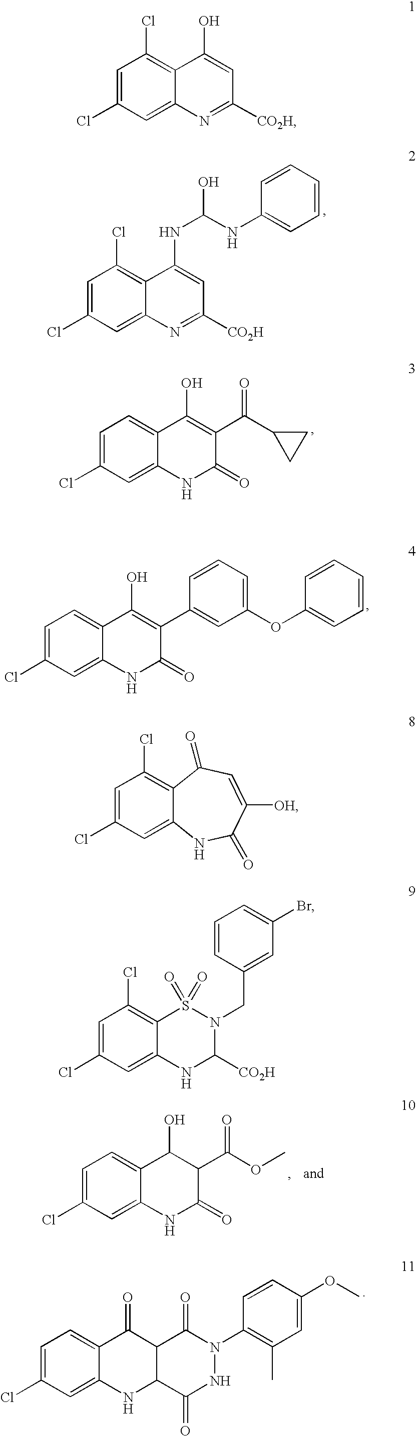

Non-limiting examples of compounds of Formula II include the following:

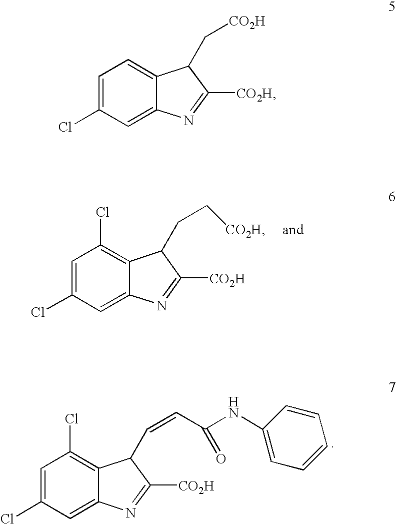

The term “halogen” is used to refer to F, Cl, Br, or I.

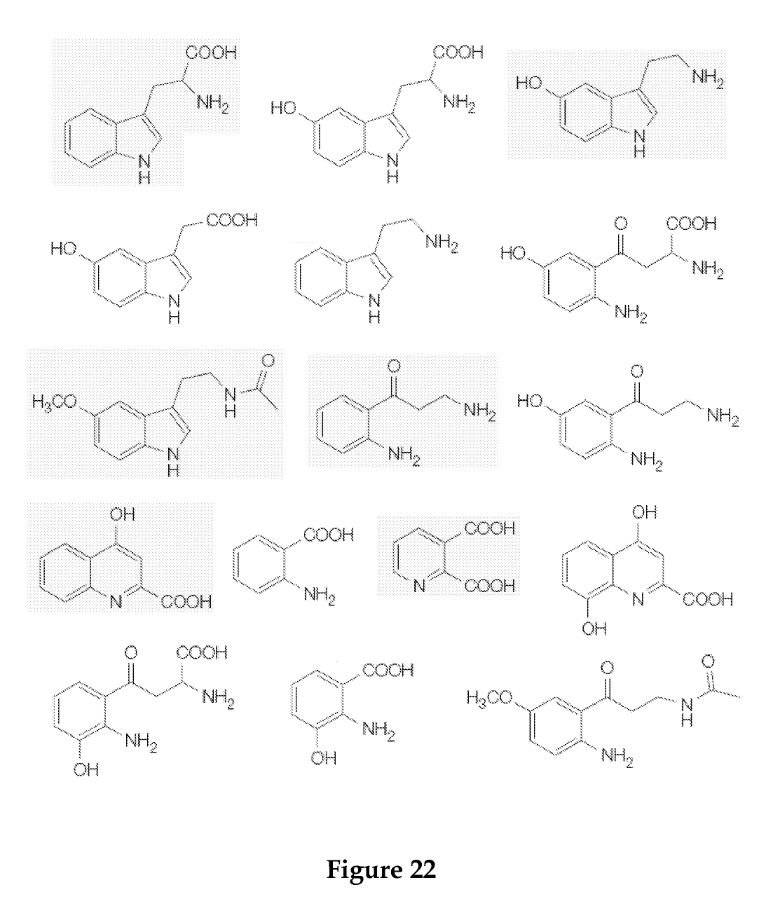

Other non-limiting examples of compounds that can be used in the disclosed methods are shown in FIG. 22.

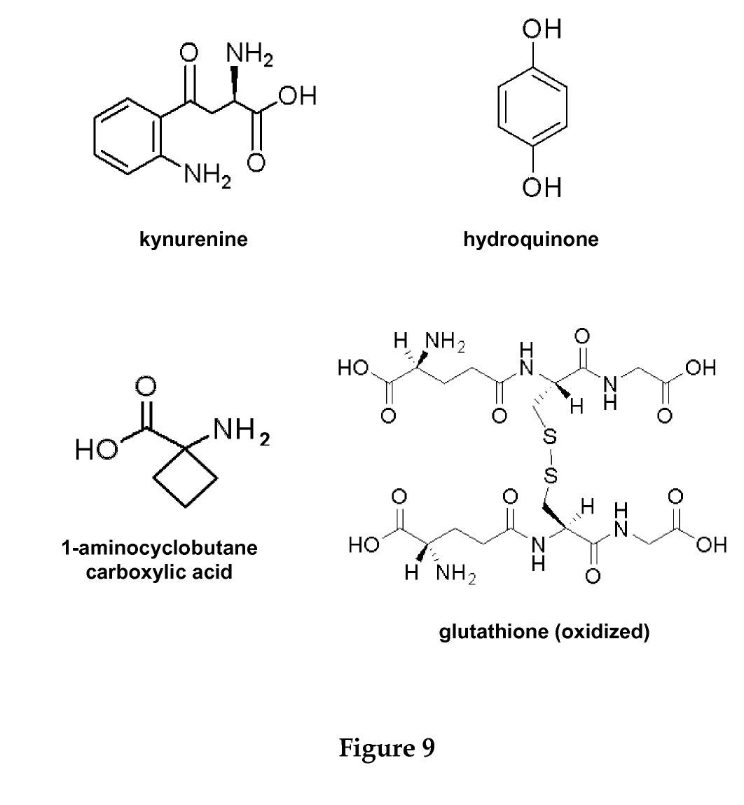

Additional non-limiting examples of compounds that can be used in the methods provided to decrease or prevent LRRK2 toxicity include compounds that inhibit GSK3β, compounds that activate AKT, or compounds that activate the AKT pathway or downstream components. Other examples of compounds include N-methyl-D-aspartic acid (NMDA) receptor antagonists or partial agonists (for example AP-5, MK-801, L-701,324, kynurenine, ACBC and hydroquinone) and antioxidants (for example, glutathione, hydroquinone and flavanone) (see FIG. 9). Other exemplary compounds include fragments of LRRK2, such as a LRRK2 kinase domain. Other examples include knockdown shRNA vectors that reduce the expression of LRRK2 in neurons, to protect neurons from process loss and death. Sequences that may be used for exemplary knockdown vectors are provided in Example 2. It is also a discovery of the invention that knock-down of LRRK2 expression, for example by shRNA, may protect neurons in PD and other neurological disorders. Therefore, the methods of the invention can be used to identify compounds that have the therapeutic potential to protect neurons in PD and other neurological disorders.

In one aspect, the invention provides for the use of PD-associated LRRK2 inhibitors to inhibit LRRK2 activity, increase axonal length, increase axonal branching, or any combination thereof in a neuronal cell. Accordingly, the invention provides a method for inhibiting activity of a Parkinson's disease-associated LRRK2 mutant protein in a neuronal cell, the method comprising contacting the cell with an NMDA receptor antagonist, an antioxidant, or a compound selected from the group consisting of: 2-amino-5-phosphonopentanoate (AP-5), MK-801, L-701,324, kynurenine, 1-aminocyclobutane carboxylic acid (ACBC), hydroquinone, and glutathione.

An example of a compound that can be used within context of the disclosed methods, include a nucleic acid, or a polypeptide expressed therefrom, capable of inhibiting expression of a LRRK2 protein. The nucleic acid may comprise RNA, antisense RNA, small interfering RNA (siRNA), double stranded RNA (dsRNA), short hairpin RNA (shRNA), cDNA, DNA, or any combination thereof. Knock-down nucleic acids can be used to reduce the expression of LRRK2 in neurons, including midbrain dopamine neurons, to protect neurons from process loss and death. Knock-down of LRRK2 may be used to protect neurons in PD and other neurological disorders. An example of an shRNA construct provided by the invention targets bases 4789-4809 of the rodent LRRK2 gene with GenBank Accession No. NM_{—}025730 (SEQ ID NO:3).

Another example is a compound comprising a peptide fragment of a LRRK2 protein. For example, the compound can comprise a LRRK2 kinase domain, consist essentially of a LRRK2 kinase domain, or consist of a LRRK2 kinase domain.

As the PTEN/PI3K/AKT/Gsk3β signal cascade plays a central role in the regulation of neuronal length and complexity, and also functions downstream of glutamate excitotoxicity, the interaction between LRRK2 and AKT/GSK3β was investigated using the disclosed methods. AKT activation or GSK3β suppression inhibit the toxicity of LRRK2. LRRK2 and AKT both lead to altered process length and the invention provides that the two ‘co-suppress’ one another. This shows that they may converge on the same point in a signal transduction pathway (or each function at multiple points along a pathway). LRRK2 and AKT both ultimately regulate survival in primary neurons. It is a discovery of the invention that glutamate excitotoxicity is involved in the phenotype of LRRK2 mutant cells, and that the AKT/GSK3β pathway impinges on LRRK2 toxicity and can suppress it. Thus, a compound used in the disclosed methods inhibits glycogen synthase kinase 3 beta (GSK3β) or activates an AKT (protein kinase B) protein, downstream components in an AKT pathway, or both.

In some embodiments, the compound can be combined with a carrier. The term “carrier” is used herein to refer to a pharmaceutically acceptable vehicle for a pharmacologically active agent. The carrier facilitates delivery of the active agent to the target site without terminating the function of the agent. Non-limiting examples of suitable forms of the carrier include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration.

Pharmaceutical formulations include those suitable for oral or parenteral (including intramuscular, subcutaneous and intravenous) administration. Forms suitable for parenteral administration also include forms suitable for administration by inhalation or insufflation or for nasal, or topical administration. The formulations may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

### Methods of Treatment

Methods are provided for treating, preventing, delaying the onset or progression of, or alleviating symptoms of a neurodegenerative disease in a subject by administering an effective amount of a compound provided herein.

High-throughput assays are provided to identify drug candidate compounds that inhibit the activity of PD-associated LRRK2 mutant proteins, thereby indicating that these drug candidates may be potential therapeutic agents for PD and other neurodegenerative diseases. A compound identified using the disclosed methods as an inhibitor of a PD-associated LRRK2 mutant represents a potential therapeutic compound that can be administered in an effective amount to a patient in need thereof.

Accordingly, in one aspect, a method is provided for treating a neurodegenerative disease in a subject, the method comprising administering to the subject an effective amount of a compound selected from the group consisting of: 2-amino-5-phosphonopentanoate (AP-5), MK-801, L-701,324, kynurenine, 1-aminocyclobutane carboxylic acid (ACBC), hydroquinone, and glutathione.

In another aspect, a compound of Formula I or Formula II can be used in the treatment of a neurodegenerative disease.

Nonlimiting examples of neurodegenerative disease that may be treated by the disclosed methods include Alexander disease, Alper's disease, Alzheimer's disease, Amyotrophic lateral sclerosis, Ataxia telangiectasia, Batten disease (also known as Spielmeyer-Vogt-Sjogren-Batten disease), Binswanger's disease, Bovine spongiform encephalopathy (BSE), Canavan disease, Cockayne syndrome, Corticobasal degeneration, Creutzfeldt-Jakob disease, Huntingtons disease, HIV- or AIDS-associated dementia, Kennedy's disease, Krabbe disease, Lewy body dementia, Machado-Joseph disease (Spinocerebellar ataxia type 3), Multiple sclerosis, Multiple System Atrophy, Myasthenia gravis, sporadic Parkinson's disease, autosomal recessive early-onset Parkinson's disease, Pelizaeus-Merzbacher Disease, Pick's disease, Primary lateral sclerosis, Refsum's disease, Sandhoff disease, Schilder's disease, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Stroke, Tabes dorsalis, Angelman syndrome, Autism, Fetal Alcohol syndrome, Fragile X syndrome, Tourette's syndrome, Prader-Willi syndrome, Sex Chromosome Aneuploidy in Males and in Females, William's syndrome, Smith-Magenis syndrome, 22q Deletion, and any combination thereof.

In one embodiment, the compound is administered directly into the brain of a subject. The compound can be directly administered to any structure in the brain. In one embodiment, the compound is administered to brain structures selected from the group consisting of ventral midbrain, substantia nigra, hippocampus, striatum, and cortex. In some embodiments the compound can be administered directly to a site of therapeutic interest in a subject, for example, an organ, tissue or cell of the subject, for example, brain, spinal cord or neurons, including motor neurons or dopamine neurons. In other embodiments, the compound comprises a carrier or signal which directs the compound to an organ, tissue or cell of the subject. The compound may be administered by any route known in the art including oral, intravenous, parenteral, intracerebral, intraperitoneal, intraspinal, topical, subcutaneous or inhalation.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “effective” is used herein to indicate that the inhibitor is administered in an amount and at an interval that results in the desired treatment or improvement in the disorder or condition being treated. For example, an amount effective to arrest, delay or reverse the progression of a neurodegenerative disease.

In some embodiments, nonlimiting examples of the subject include: human, mouse, rabbit, monkey, rat, bovine, pig or dog.

### Compound Screening Methods

Expression of LRRK2 mutants, such as G2019S or 12020T, leads to accumulation of neuronal inclusions, for example, axonal spheroids that stain positively for Tau protein. These spheroids resemble findings in patients with LRRK2-associated PD. Whereas AKT activation suppresses the process and survival phenotypes of LRRK2 mutant expression, it does not suppress the spheroid inclusion phenotype. Thus, Tau-positive spheroid accumulation appears to be separate from neuronal survival.

Depleting LRRK2, for example, using RNAi or inhibiting LRRK2 action with a dominant negative allele, leads to a dramatic increase in neurite length and complexity in primary cortical neurons. Consistent with this, clinical mutations in LRRK2, which disinhibit kinase activity, induce shorter and less complex processes. LRRK2 kinase domain is responsible for this activity. It is a discovery of the invention that LRRK2 regulates the maintenance and morphology, rather than the establishment, of neuronal processes.

A method is provided for determining whether a compound inhibits mutant LRRK2 protein activity, the method comprising (a) expressing in a primary neuronal cell a Parkinson's Disease-associated LRRK2 mutant protein, wherein expression of the mutant results in accumulation of axonal spheroid inclusions that stain positive for Tau protein; (b) contacting the neuronal cell with a compound; and (c) determining whether accumulation of axonal spheroid inclusions in the neuronal cell is reduced compared to accumulation of axonal spheroid inclusions in a neuronal cell expressing the LRRK2 mutant in the absence of the compound, wherein determination of a reduction in (c) indicates that the compound inhibits the LRRK2 mutant protein activity. Tau protein can be detected, for example, by immunostaining with a Tau-specific antibody or a phospho-Tau-specific antibody.

A method is provided for determining whether a compound inhibits mutant LRRK2 protein activity, the method comprising (a) expressing in a neuronal cell a Parkinson's Disease-associated LRRK2 mutant protein, wherein expression of the mutant results in decreased axonal length; (b) contacting the neuronal cell with a compound; and (c) determining whether axonal length in the neuronal cell is increased compared to axonal length in a neuronal cell expressing the LRRK2 mutant in the absence of the compound, wherein determination of an increase in (c) indicates that the compound is capable of inhibiting the LRRK2 mutant protein activity. Axonal length can be assessed, for example, using the following parameters: the length of the longest neuronal process, the total length of all neuronal processes, and the diameter of the soma along its longest axis. Neuronal processes can be visualized by techniques known in the art, for example, by intracellular expression of a detectable protein (e.g., green fluorescent protein) or by immunostaining of an axonal marker protein using a detectable antibody, or fragment thereof, that specifically binds the axonal marker protein. Quantification of axonal length can be carried out, for example, using microscopy and computer-assisted analysis as further described in Example 2.

A method is provided for determining whether a compound inhibits mutant LRRK2 protein activity, the method comprising (a) expressing in a primary neuronal cell a Parkinson's Disease-associated LRRK2 mutant, wherein expression of the mutant results in decreased axonal branching; (b) contacting the neuronal cell with a compound; and (c) determining whether axonal branching in the neuronal cell is increased compared to axonal branching in a neuronal cell expressing the LRRK2 mutant in the absence of the compound, wherein determination of an increase in (c) indicates that the compound inhibits the LRRK2 mutant protein activity. Branch points may be counted, for example, in the longest neuronal process. Neuronal processes can be visualized by techniques known in the art, for example, by intracellular expression of a detectable protein (e.g., green fluorescent protein) or by immunostaining of an axonal marker protein using a detectable antibody, or fragment thereof, that specifically binds the axonal marker protein. Quantification of axonal branching can be carried out, for example, using microscopy and computer-assisted analysis as further described in Example 2.

A method is provided for determining whether a compound inhibits mutant LRRK2 protein activity, the method comprising (a) expressing in a primary neuronal cell a Parkinson's Disease-associated LRRK2 mutant, wherein expression of the mutant results in reduced survival of the neuronal cell; (b) contacting the neuronal cell with a compound; and (c) determining whether survival of the neuronal cell is increased compared to survival of a neuronal cell expressing the LRRK2 mutant in the absence of the compound, wherein determination of an increase in (c) indicates that the compound inhibits the LRRK2 mutant protein activity.

In one embodiment, the LRRK2 mutant protein expressed by the primary neuronal cell consists essentially of a LRRK2 kinase domain, wherein the kinase domain comprises one or more Parkinson's Disease-related LRRK2 mutations. In other embodiments, the LRRK2 mutant protein comprises a G2019S mutation, an 12020T mutation, or both. The locations of the LRRK2 mutations described herein are based on the amino acid sequence (SEQ ID NO:1) translated from the mRNA sequence of human LRRK2 (SEQ ID NO:2) (GenBank Accession No. AY792551).

In one embodiment, the primary neuronal cell is in a cell culture. In another embodiment, the primary neuronal cell is in vivo in an animal. In another embodiment, the primary neuronal cell is a post-mitotic neuron. In another embodiment, the post-mitotic neuron is a cortical neuron, a dopamine neuron, or a sympathetic neuron.

A primary neuronal cell used within the context of the invention may comprise a nucleic acid vector encoding a Parkinson's Disease-related LRRK2 mutant. In another embodiment, the vector comprises a nucleic acid sequence encoding a fragment of a LRRK2 protein. In another embodiment, the vector comprises a nucleic acid sequence encoding a LRRK2 kinase domain. An example of a nucleic acid vector is a viral vector. Non-limiting examples of viral vectors include a lentiviral vector, an adeno-associated virus-2 (AAV-2) vector, an adenoviral vector, a retroviral vector, a polio viral vector, a murine Maloney-based viral vector, an alpha viral vector, a pox viral vector, a herpes viral vector, a vaccinia viral vector, a baculoviral vector, a parvoviral vector, or any combination thereof. In one aspect, the invention provides a viral vector comprising a nucleic acid encoding a LRRK2 kinase domain, or a fragment thereof, wherein the kinase domain comprises one or more Parkinson's Disease-associated LRRK2 mutations.

Other embodiments of the disclosed methods comprise expressing a fluorescent protein in the primary neuronal cell. The expression of a fluorescent protein, such as green fluorescent protein (GFP), enables visualization of neuronal cells and neuronal processes. In one embodiment, the determining comprises detecting fluorescence. Fluorescence can be detected directly (e.g., detection of GFP) or indirectly (e.g., detection of an antibody with a fluorescent label or tag, wherein the antibody specifically binds the protein of interest, such as LRRK2 or an axonal marker protein). In another embodiment, the determining comprises computer-assisted quantification of axonal length. In another embodiment, the determining comprises computer-assisted quantification of axonal branching. An example of a computer-assisted technique for quantitative analysis of axonal morphology is described in Example 2.

The disclosed methods can be carried out in a multi-well plate. The methods can be carried out in a high-throughput manner. In another embodiment, the method is carried out for more than one hundred compounds.

### In Vivo Screening Methods

The screening methods, as described above, can be carried out in a neuronal cell in vivo in an animal to develop novel animal models of PD. For example, methods are provided for in vivo infection of adult rat midbrain dopamine neurons using an AAV2-based viral vector. Using the disclosed methods, mutant LRRK2 expression was shown to lead to early axonal inclusions, process loss, and increased apoptosis in midbrain dopamine neurons. An animal model of LRRK2-associated Parkinsonism is described in Example 1.

A second animal model is provided based on in utero electroporation, that allows for the study of cell-autonomous changes in single neurons in the intact CNS. A novel in utero gene transduction assay, which was used to show that LRRK2 also regulates process morphology in a cell autonomous manner in the intact brain.

The following examples illustrate the present invention, and are set forth to aid in the understanding of the invention, and should not be construed to limit in any way the scope of the invention as defined in the claims which follow thereafter.

### EXAMPLES

### Example 1

### The Familial Parkinsonism Gene LRRK2 Regulates Neuronal Process Morphology and Maintenance

Mutations in LRRK2 underlie an autosomal dominant, inherited form of Parkinson's disease (PD) that mimics all of the clinical features of the common sporadic form of PD. The LRRK2 protein includes putative GTPase, protein kinase, WD40 repeat, and leucine rich repeat (LRR) domains of unknown function. This Example shows that PD-associated LRRK2 mutations display disinhibited kinase activity and induce a progressive decrease in neurite length and branching both in primary neuronal cultures and in the intact rodent CNS. In contrast, LRRK2 deficiency leads to increased neurite length and branching. Neurons that express PD-associated LRRK2 mutations additionally display prominent lysosomal abnormalities and Tau-positive neuritic inclusions. PD-associated LRRK2 pathology is ameliorated by NMDA receptor antagonists and antioxidants, consistent with a role for oxidative stress and glutamate excitotoxicity in the disease mechanism.

This Example shows that mammalian LRRK2 regulates neurite maintenance and neuronal survival. LRRK2 protein is associated with membranous neuronal compartments, and an early feature of cells expressing Parkinsonism-associated LRRK2 mutants is the prominent accumulation of abnormal swollen lysosomes and mitochondria. Neurons that express disease-associated mutant forms of LRRK2 also display reduced process length and complexity, Tau-positive protein aggregates, and, ultimately, apoptotic cell death. In contrast, neurons deficient in LRRK2 harbor extended axonal and dendritic processes and display increased branching. NMDA receptor antagonists and antioxidants inhibit LRRK2 mutant-associated phenotype, consistent with a role for glutamate excitotoxicity and showing potential therapeutic strategies.

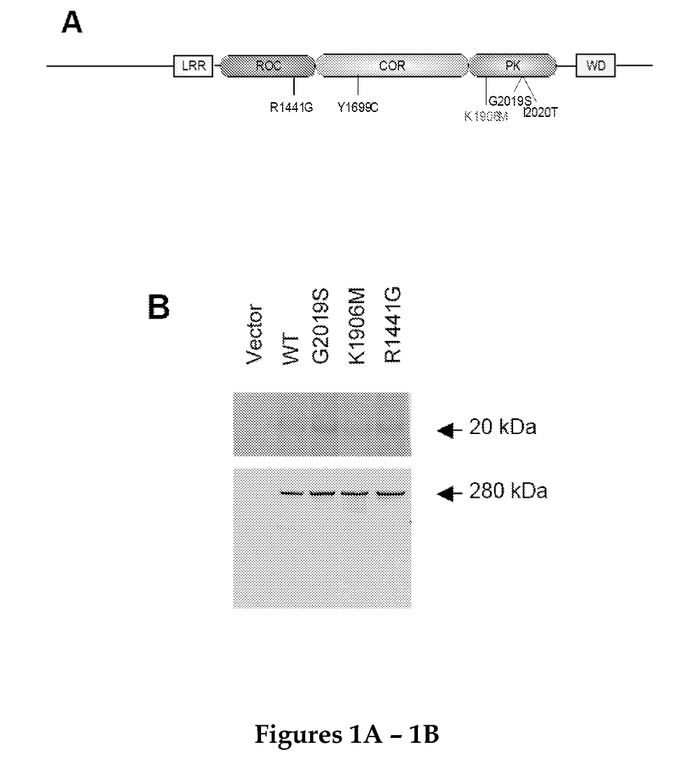

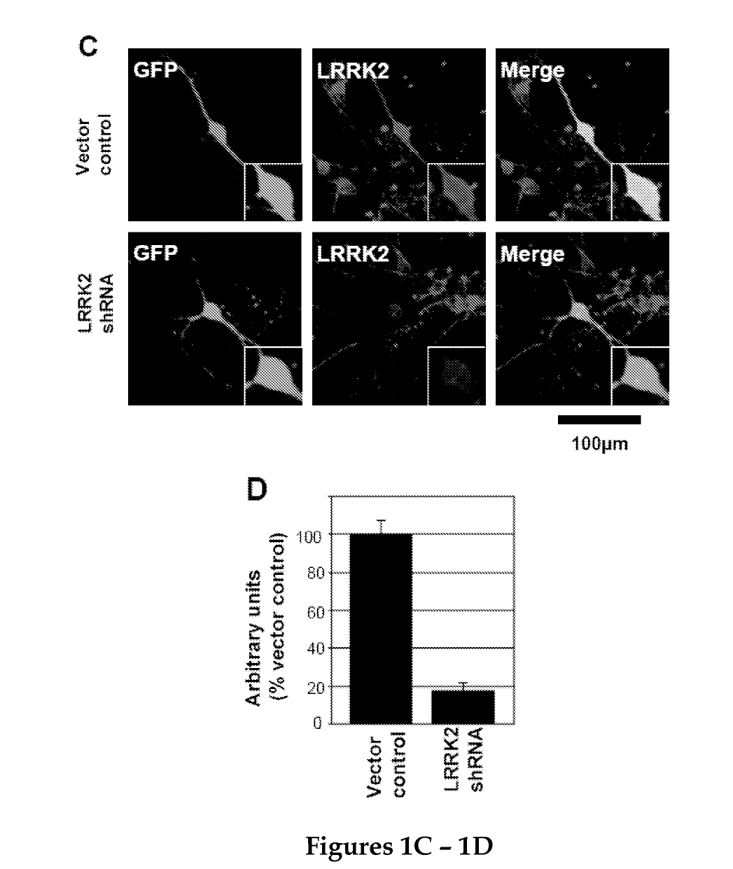

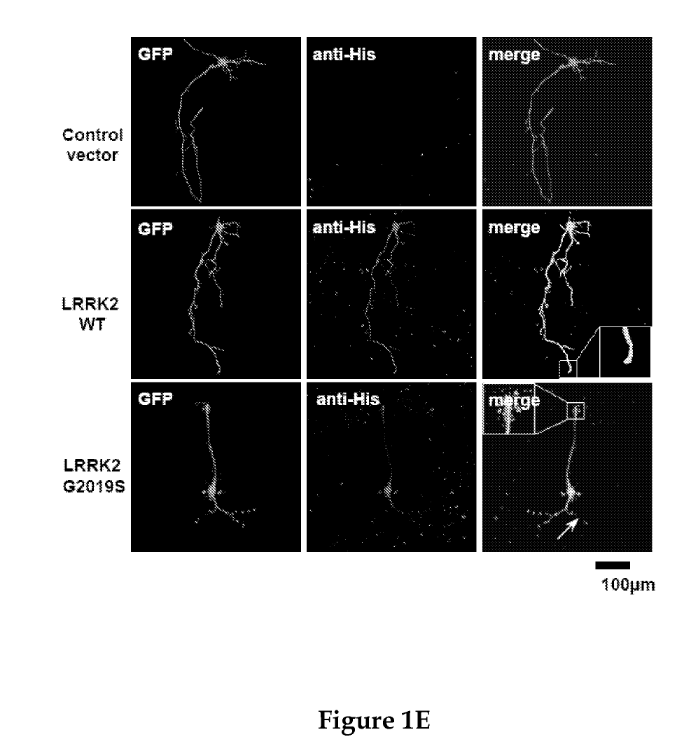

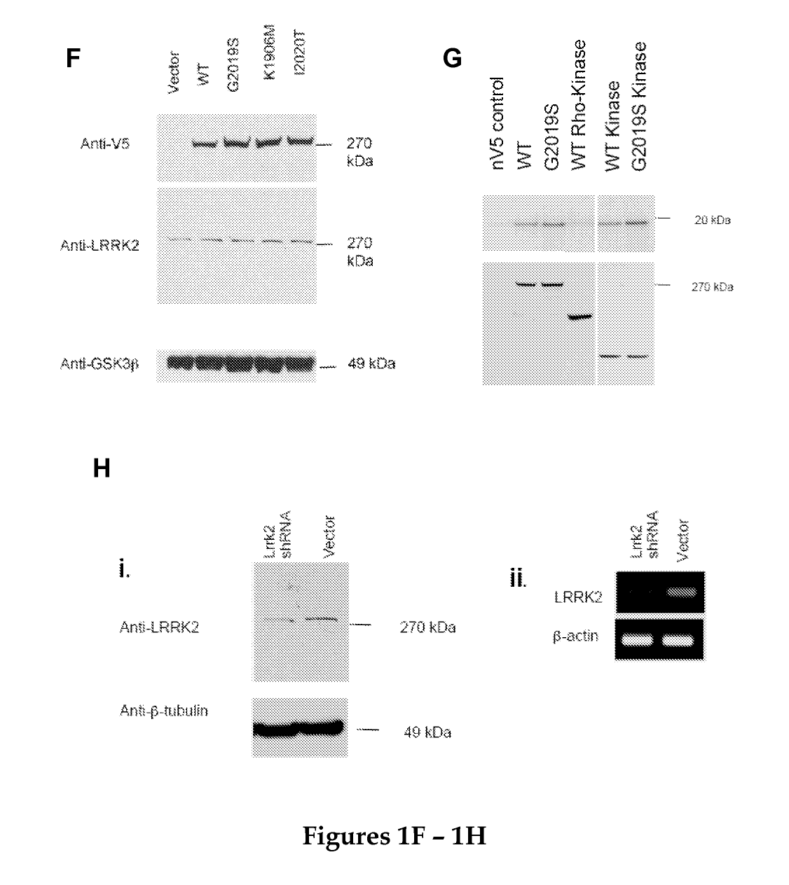

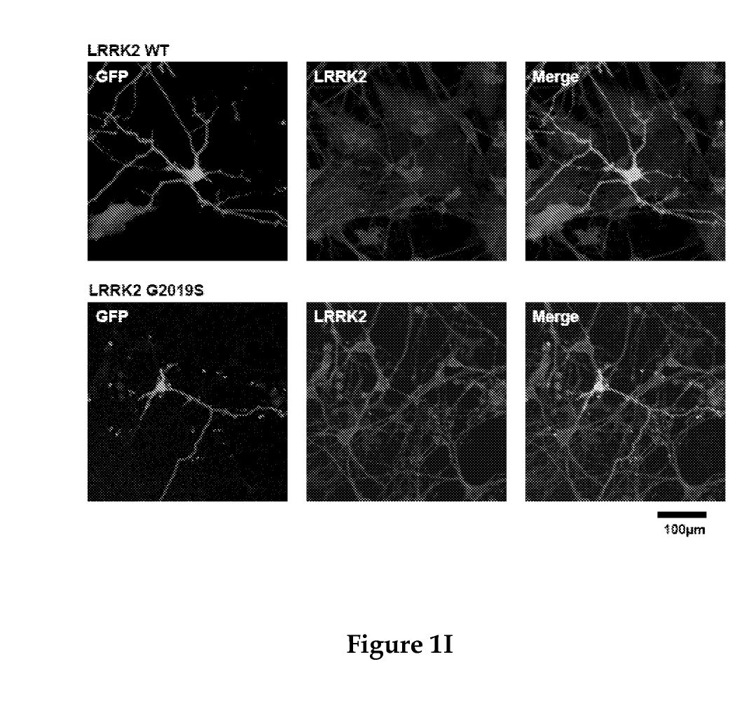

LRRK2 expression and kinase activity. To investigate the normal and pathological functions of LRRK2, the invention provides plasmid vectors for overexpression of wild-type or disease-associated mutant alleles of LRRK2 including G2019S, I2020T, Y1699C, and R1441G (FIGS. 1A and 1B). A V5/His-epitope tag was added to the amino terminus of each coding sequence to distinguish the plasmid-encoded protein from endogenous LRRK2. Plasmids were transiently transfected into either COS7 or 293T cells, and cell lysates were analyzed by SDS-PAGE and Western blotting with antibodies recognizing the V5-epitope tag (FIG. 1B) or LRRK2 (FIG. 1F). A single 280 kDa protein was observed in cytoplasmic lysates of cells transfected with either wild-type or mutant LRRK2 plasmids, and transfection of LRRK2 plasmids led to approximately 2-fold increased expression over the endogenous level (FIG. 1F).

Cell lysates from transfected cells were immunoprecipitated using an antibody for the V5 epitope tag, and subsequently the immunoprecipitated complexes were assayed for kinase activity towards purified myelin basic protein (MBP) or myosin light chain (MLC), common substrates for mixed-lineage kinase related proteins. Wild-type LRRK2 displayed a low level of kinase activity in this assay, whereas the G2019S mutation led to significantly increased kinase activity towards both MLC (FIG. 1B) and MBP (FIG. 1G). These data are consistent with the hypothesis that the G2019S mutation leads to disinhibited kinase activity relative to wild type LRRK2.

Additionally, the invention provides two short hairpin RNA (shRNA)-based plasmid vectors were generated to inhibit the expression of endogenous rodent LRRK2 by RNA interference (RNAi). Transfection of either of these plasmids into primary rat cortical cultures or rat C6 glioma cells led to a reduction in the level of LRRK2 mRNA and protein to less than 20% of baseline levels, as determined by real-time quantitative rtPCR, Western blotting, and immunohistochemistry with polyclonal antibodies for LRRK2 (FIGS. 1C, 1D and 1H). Immunohistochemical analysis of primary cortical cultures further revealed the presence of LRRK2 throughout the soma and neurite processes of neurons (FIG. 1C). Prominent staining is associated with intracellular membrane compartments, consistent with biochemical analysis of LRRK2 localization (Gloeckner et al., 2006). Primary cortical cultures were transfected with plasmid vectors encoding wild-type or mutant forms of V5 epitope-tagged LRRK2 (or vector control), along with enhanced green fluorescent protein (eGFP) sequences to allow for the identification of transfected cells (approximately 5% of neurons). Immunohistochemistry with an antibody for the epitope-tag (FIG. 1E) or with an antibody for LRRK2 (FIG. 1I) indicated that exogenous wild-type or mutant alleles of LRRK2 localized similarly to the endogenous protein. The G2019S PD-associated LRRK2 mutants were also present in distinctive spheroid-like inclusions within cellular processes and at intracellular membranous structures (see below).

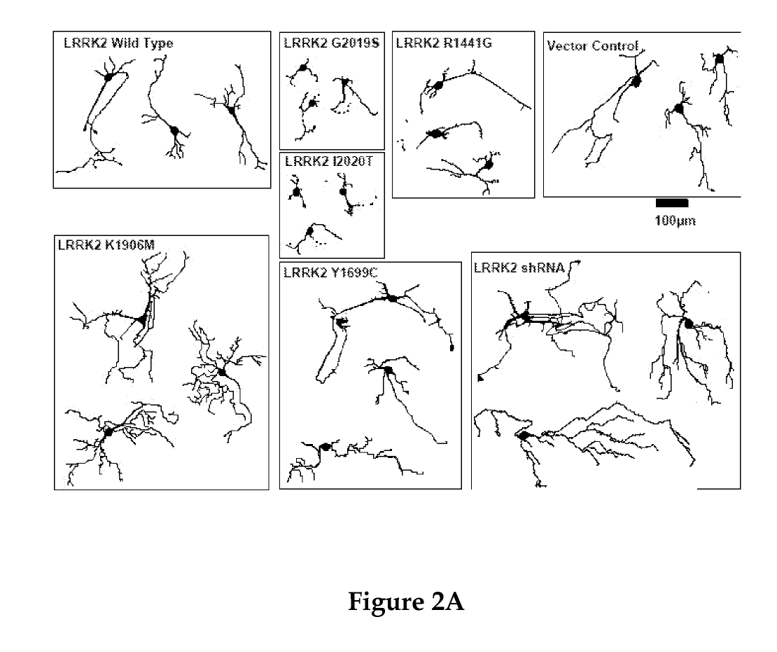

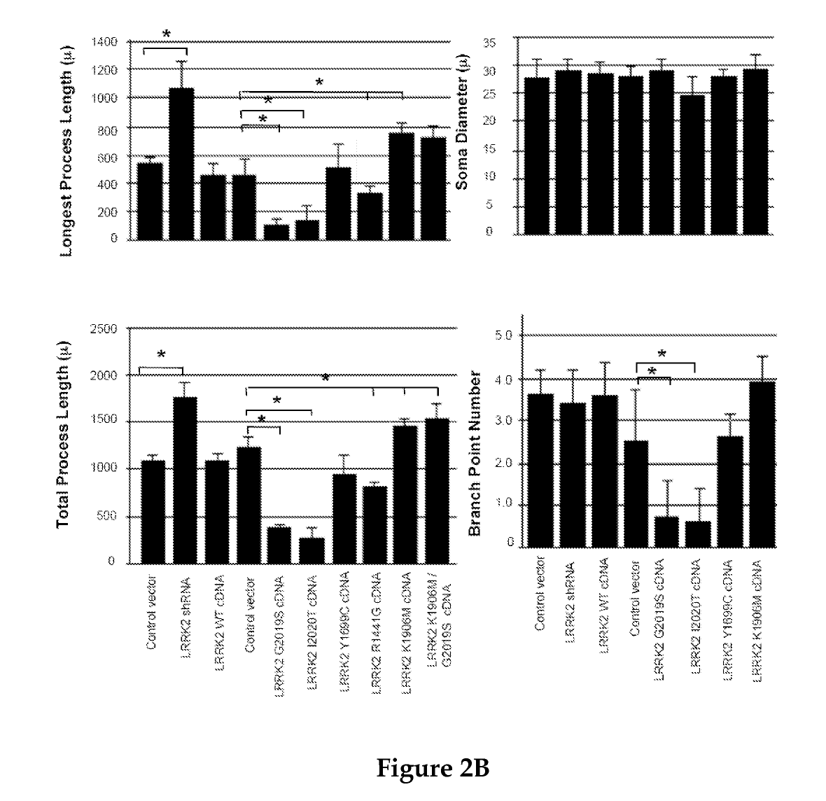

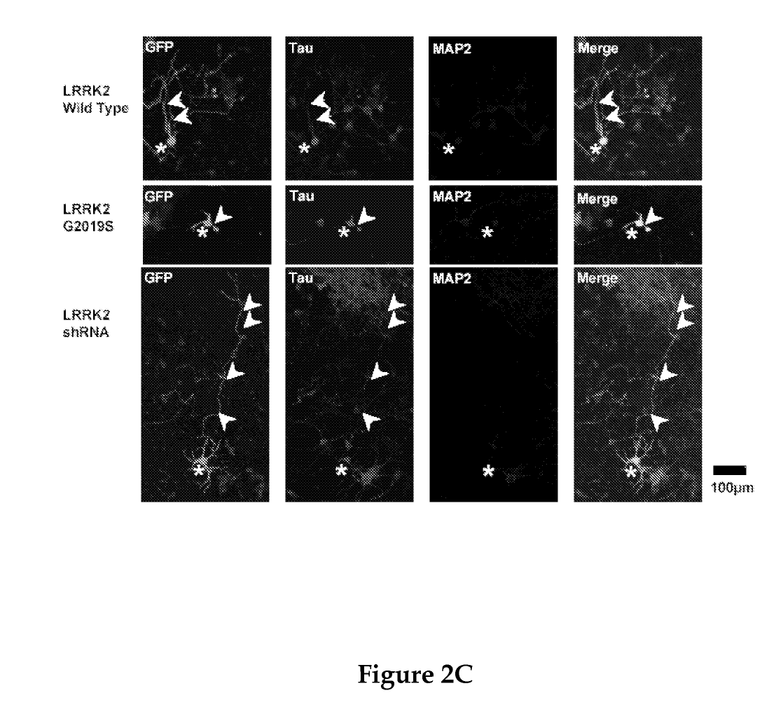

LRRK2 regulates neuronal process morphology. Primary cortical neurons expressing Parkinsonism-associated mutant alleles of LRRK2 appeared to display reduced neuron processes (FIG. 1E). Overexpression of either of two clinically-associated missense mutant forms of LRRK2 within the kinase domain, G2019S and 12020T, led to a dramatic reduction in neurite length and branching evident with respect to both the longest neuronal processes, corresponding to axons, as well as dendritic processes (FIGS. 2A and 2B), confirmed by antibody staining for axonal and dendritic markers (FIG. 2C). Neuronal polarity, as quantified by the ratio of axons to dendrites, appeared unaltered. Overexpression of a Parkinsonism-associated missense mutation in the ROC domain, R1441G, also led to a significant decrease in process length, whereas a mutation within the COR domain, Y1699C, induced a relatively modest decrease in process length that did not reach statistical significance (FIGS. 2A and 2B). Overexpression of wild-type LRRK2 did not alter neuronal morphology, and the soma size of neurons transfected with either wild-type (WT) or mutant LRRK2 allele cDNA appeared similar to vector control.

As Parkinsonism-associated LRRK2 alleles display disinhibited kinase activity and short processes, experiments were designed to test whether neurons deficient in LRRK2 activity may demonstrate extended processes. An additional mutant form of LRRK2, K1906M, was generated that is predicted to lie within the ATP binding segment of the kinase domain (Cobb and Goldsmith, 1995) and has been shown to generate a dominant negative allele (Cobb and Goldsmith, 1995; Gloeckner et al., 2005). Overexpression of the K1906M allele led to a significant increase in total process length, as effect that was particularly evident with respect to the length of the longest process (FIGS. 2A and 2B). In a second approach, LRRK2 accumulation was inhibited by RNA interference. Cortical neurons transfected with either of the two shRNA vectors specific for LRRK2 displayed a prominent increase in neurite length (FIGS. 2A and 2B).

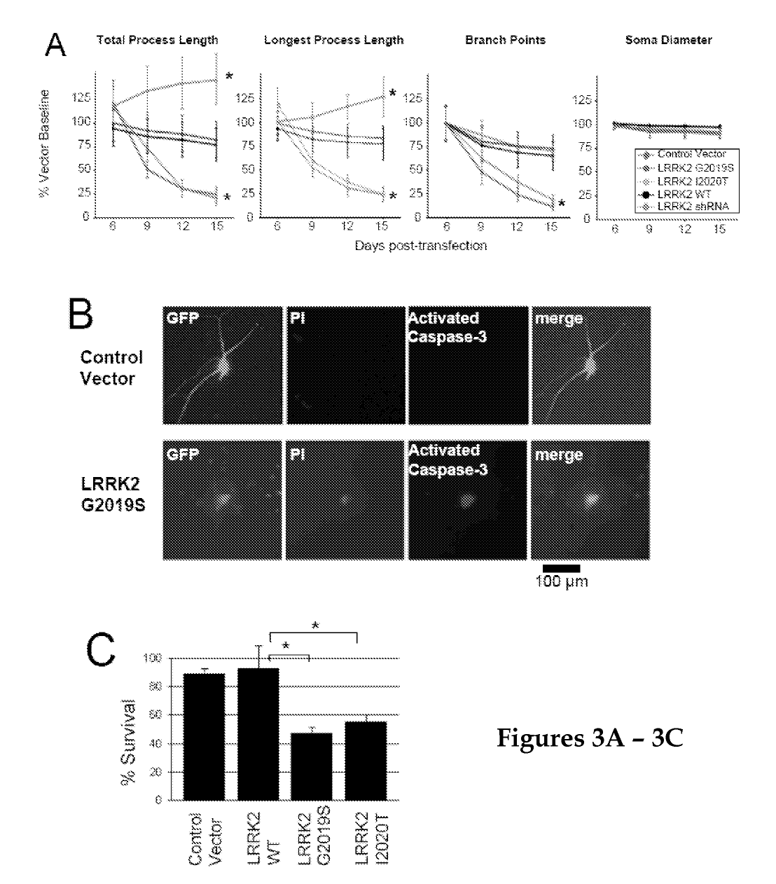

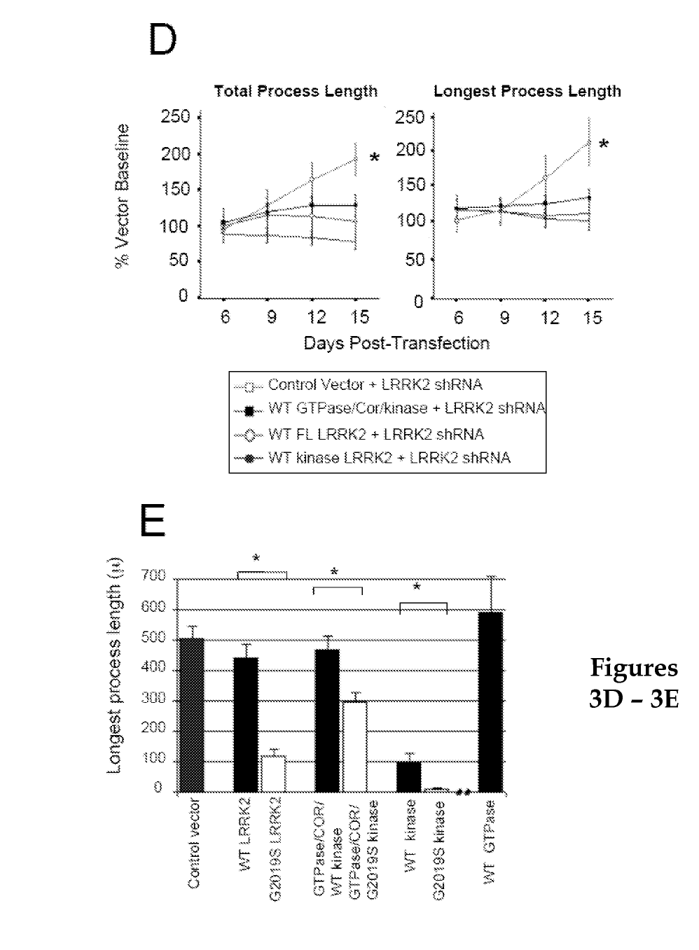

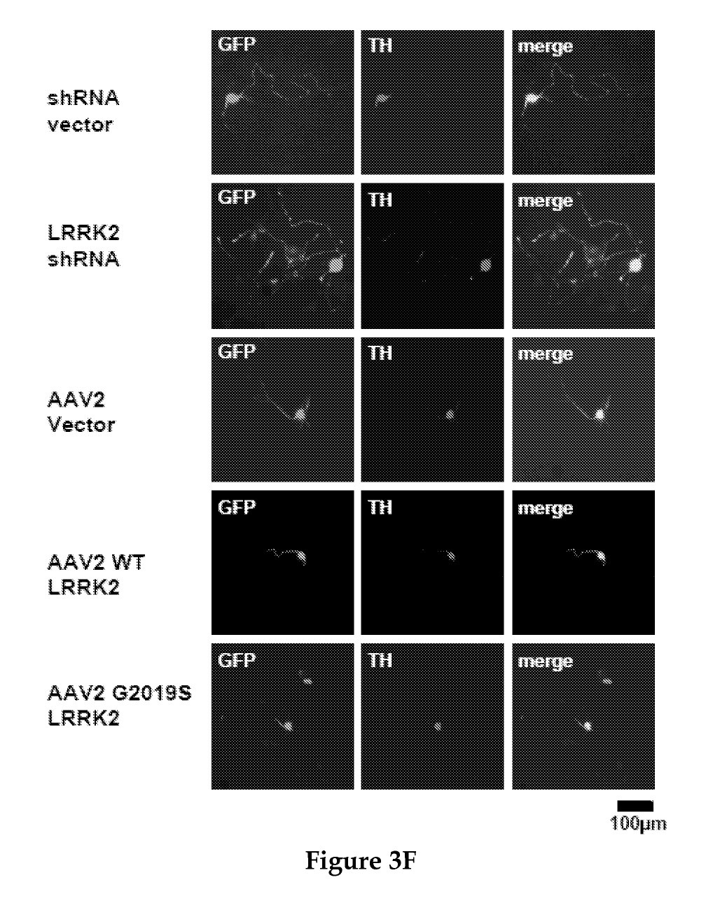

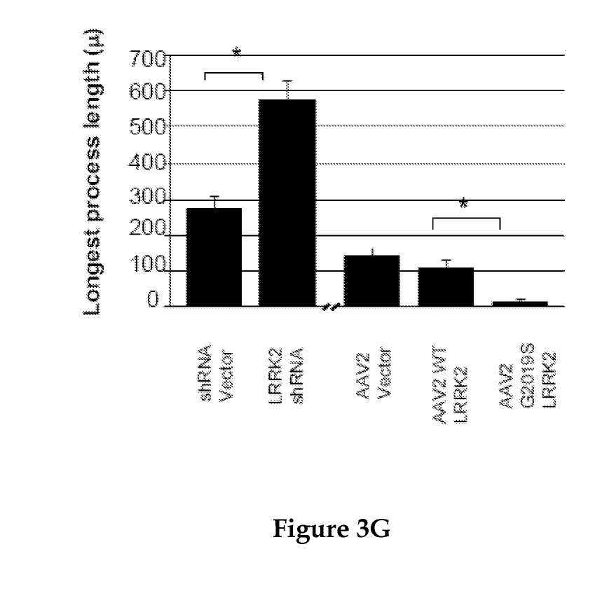

LRRK2 mutations and the maintenance of neuronal processes. To distinguish between a role in the generation and the maintenance of process length, a time course analysis of neuronal morphology was performed in cortical cultures transfected with LRRK2 wild-type, G2019S, or I2020T overexpression plasmids, or with the LRRK2 knockdown shRNA vector. Cultures were transfected as above and then individual cells were followed by fluorescence microscopy at 6, 9, 12, and 15 days subsequently. This time course analysis demonstrated that overexpression of PD-associated LRRK2 mutants G2019S or I2020T, but not wild-type LRRK2, led to a progressive decline in the length of processes (FIG. 3A). Furthermore, LRRK2 disease-associated mutations were found to display a progressive reduction in branch points emanating from the axon. Soma diameter was not significantly reduced in the mutant LRRK2 transfected neurons. Time course analysis of LRRK2 knockdown in cortical neuron cultures indicated a gradual and progressive increase in total process length (FIG. 3A).

At late time points, decreased neuron survival was evident in neurons that express the Parkinsonism-associated LRRK2 mutant allele. By day 15 post-transfection, survival was significantly reduced in mutant G2019S LRRK2 transfected cortical neurons in comparison to wild-type or vector-only transfected cells (45% survival in the G2019S versus 90% in the wild type or vector transfected cells; P<0.05), as determined by exclusion of propidium iodide stain and nuclear condensation (FIGS. 3B and 3C). Immunohistochemistry for activated caspase-3 (FIGS. 3B and 3C) demonstrated that the mutant G2019S-transfected cells undergo an apoptotic mechanism of cell death.

Structure/function analysis of LRRK2 reveals a critical role for the kinase domain. To establish a structure/function relationship of LRRK2 domains, experiments were designed to ‘rescue’ the shRNA knockdown allele phenotype by overexpression of LRRK2 cDNA sequences. Transfection of the LRRK2 shRNA vector along with overexpression of wild-type LRRK2 sequences effectively ‘rescues’ the elongated process morphology phenotype (FIG. 3D). The kinase domain alone is sufficient for functional rescue of the knockdown shRNA phenotype. Deletion analysis revealed that the kinase domain alone is also sufficient for the shortened neurite phenotype in the context of Parkinsonism-associated LRRK2 allele expression (FIG. 3E). The kinase domain alone displays a more dramatic short-neurite phenotype than full-length LRRK2, in the context of either G2019S mutant or wild-type alleles, showing that there exist inhibitory domains within LRRK2 that negatively regulate kinase activity. The kinase domain alone displays a significant (approximately 50%) increase in kinase activity in vitro (FIG. 1G). An LRRK2 allele engineered to harbor both the G2019S clinical mutation and the K1906M kinase-dead mutation displays normal processes (FIG. 2B), confirming that kinase activity is essential for the mutant allele short-neurite phenotype. Finally, analysis of the phenotype of primary midbrain cultures showed that LRRK2 G2019S-expression leads to neurite process defects in dopamine neurons (FIGS. 3F and 3G).

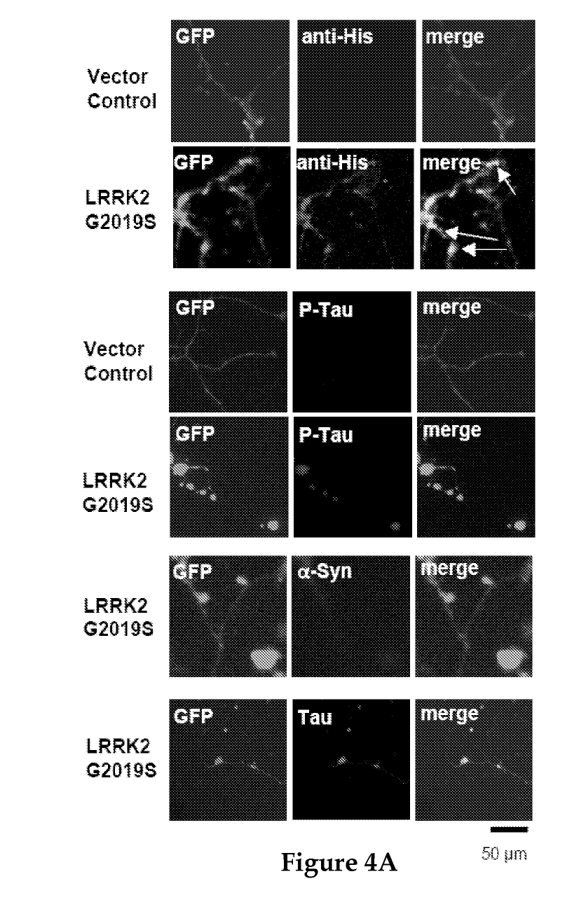

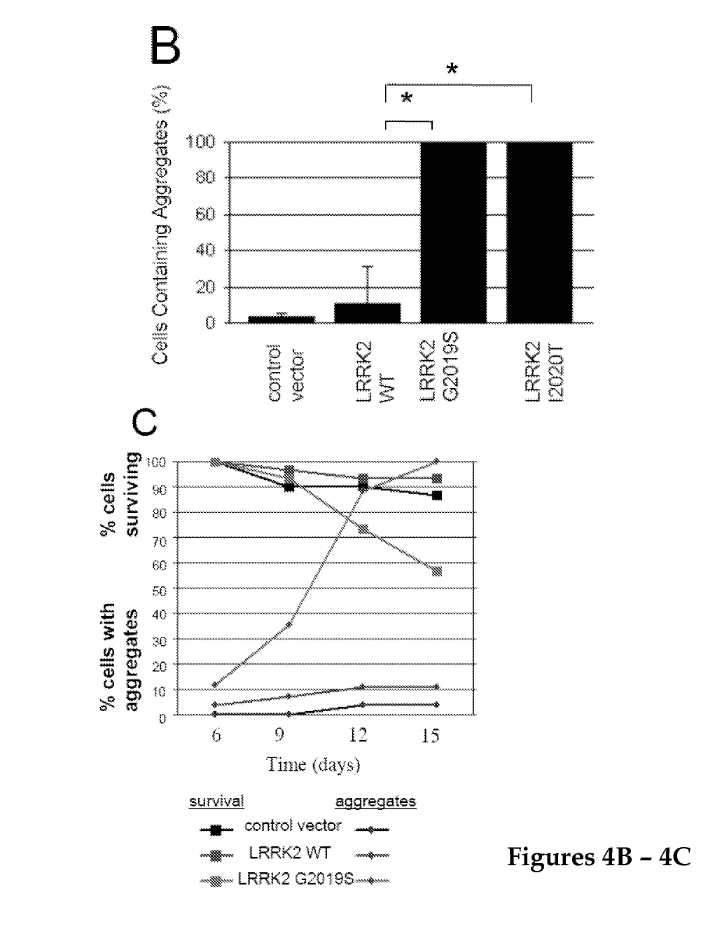

LRRK2 mutations induce Tau-positive spheroid axonal inclusions in vitro. Prominent spheroid-like aggregates were observed within the neuronal processes in all of the G2019S (37 out of 37) and I2020T (20 out of 20) transfected cortical neurons, but only rarely in vector alone (3 out of 85) or wildtype LRRK2 (4 out of 45) transfected cells, as determined by fluorescence microscopy for the eGFP marker (FIGS. 4A and 4B). The inclusions stained positively with a monoclonal antibody for the epitope-tagged G2019S LRRK2 protein (FIG. 4A). Tau protein phosphorylated at serine 202 (phospho-Tau), visualized by immunostaining with a phospho-specific antibody (FIG. 4B), as well as total Tau protein, specifically accumulated in the spheroidal inclusions. Phospho-Tau-positive axonal spheroid aggregates are described in the pathology of a number of neurodegenerative syndromes, including LRRK2-associated Parkinsonism (Wszolek et al., 2004). The spheroid aggregates did not stain positively with an antibody for α-Synuclein (FIG. 4C). Pathological specimens from patients with LRRK2-associated Parkinsonism have been found to show variable α-Synuclein pathology. A time course analysis revealed that aggregate formation parallels the neurite defect phenotype in G2019S expressing cells (FIG. 4F).

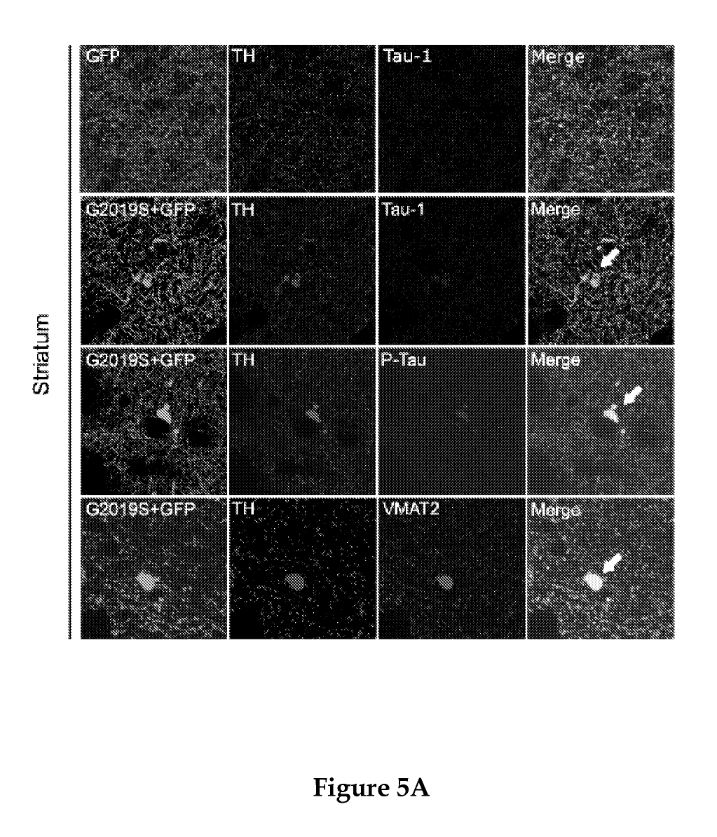

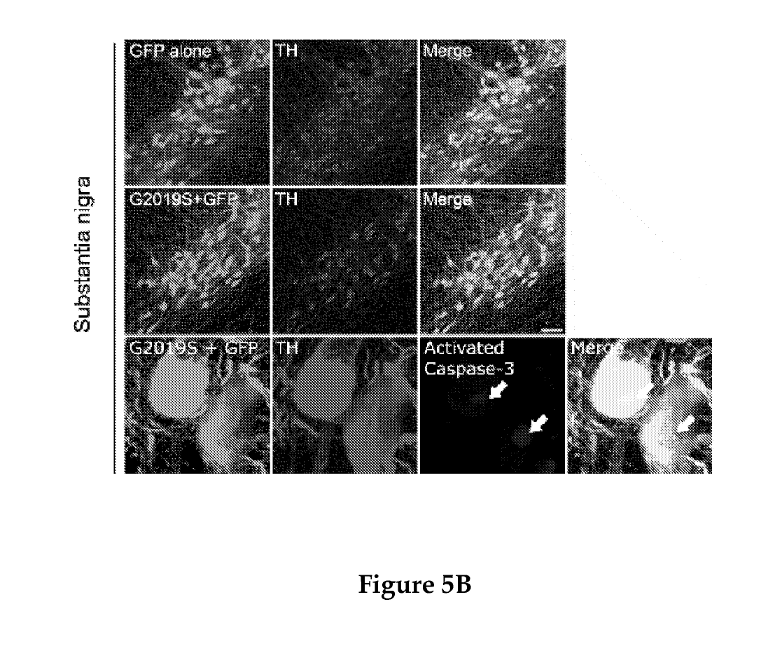

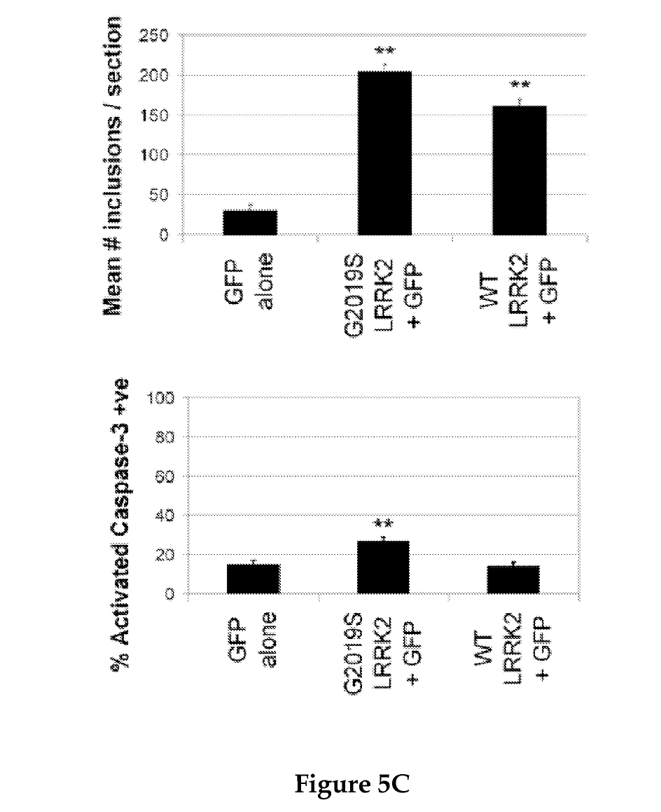

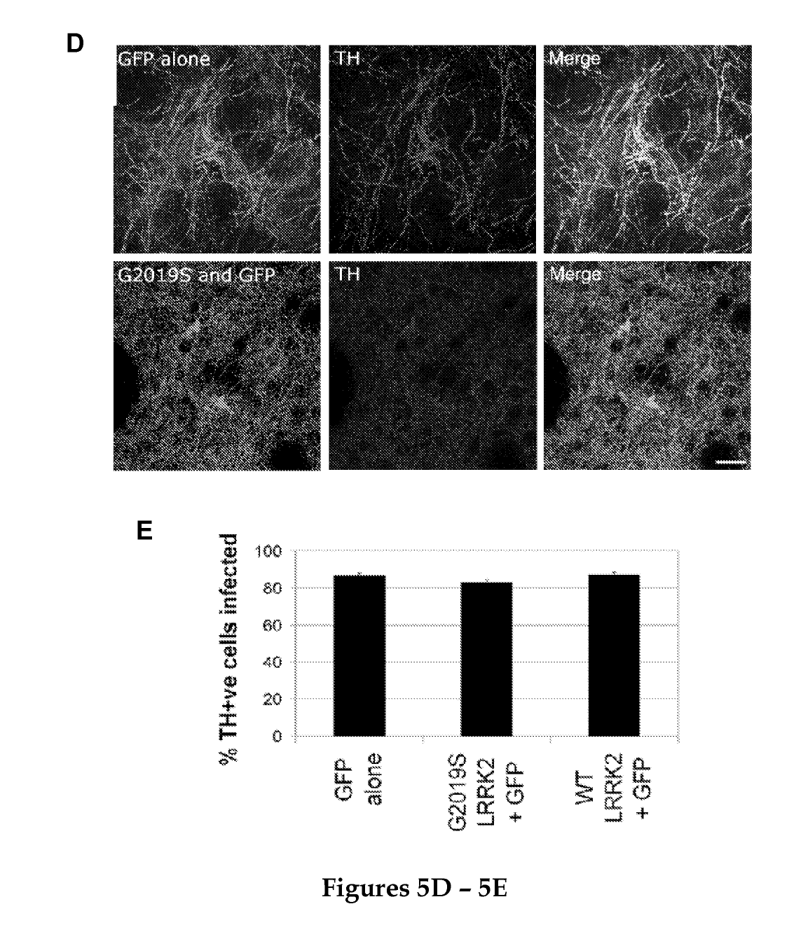

LRRK2 mutation in adult nigral dopamine neurons. Using an animal model provided by the invention, LRRK2 function was further analyzed in adult rat substantia nigra dopamine neurons (DNs) using an adeno-associated virus-2 (AAV-2) mediated gene transduction model. As the kinase domain of LRRK2 is sufficient to induce the G2019S-associated cellular phenotypes in vitro (FIG. 3E), and because of the genome size limitation of viral vectors, the kinase domain alone of either wild-type or G2019S mutant LRRK2 was overexpressed. AAV2 vectors were stereotactically injected into the substantia nigra pars compacta within the ventral midbrain of young adult rats along with GFP vector to allow visualization of transduced cells. After 1 month, rats were sacrificed and pathological examination of the brain was performed. All analyses were performed by an observer blinded to the genotype. In the context of G2019S overexpression, and with wild-type LRRK2 overexpression to a lesser extent, dopaminergic axonal processes extending into the striatum displayed prominent abnormal morphology and inclusions (as identified by GFP expression and TH immunostaining; FIG. 5, n=8 for each group), consistent with the in vitro phenotype. Immunohistochemical analysis revealed that the inclusions stained positively for Tau and phospho-Tau (at serine 404), as well as for VMAT-2 (a dopamine vesicle terminal marker), but not for α-Synuclein (FIGS. 5A and 5B). Dopamine neurons in the substantia nigra appeared grossly normal in the context of G2019S LRRK2 expression, (FIGS. 5B and 5C), but apoptosis was significantly increased, as quantified by nuclear morphology and immunohistochemistry for activated caspase-3 (FIGS. 5B and 5C). Overexpression of the wild-type kinase domain alone led to the induction of some axonal inclusions, but to a lesser extent than the G2019S mutant.

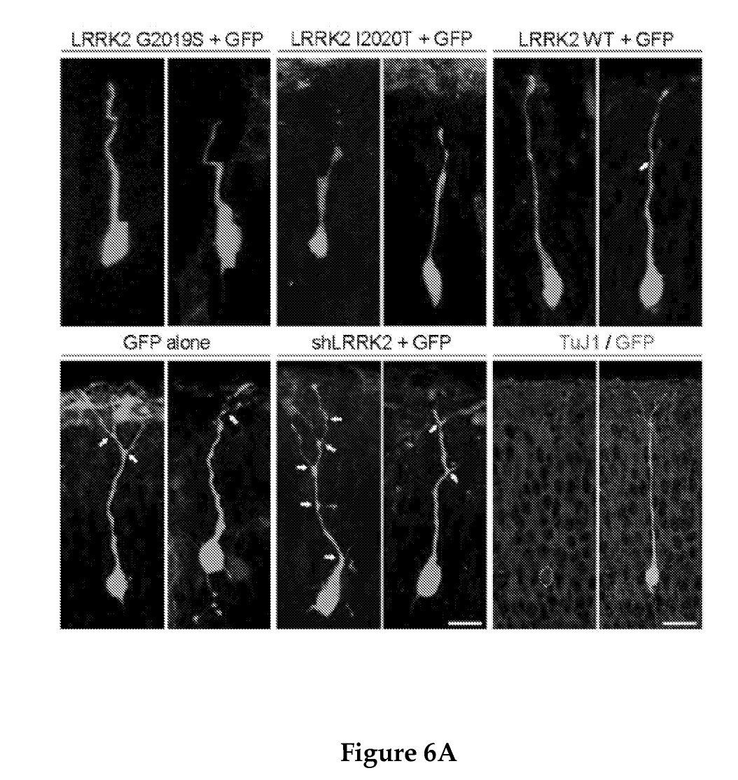

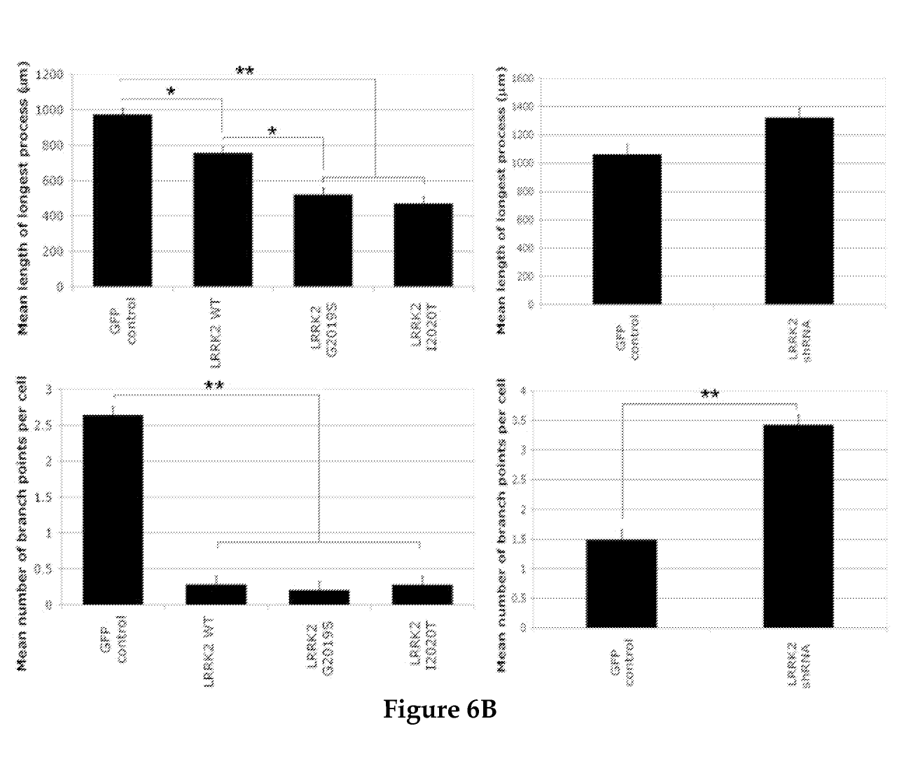

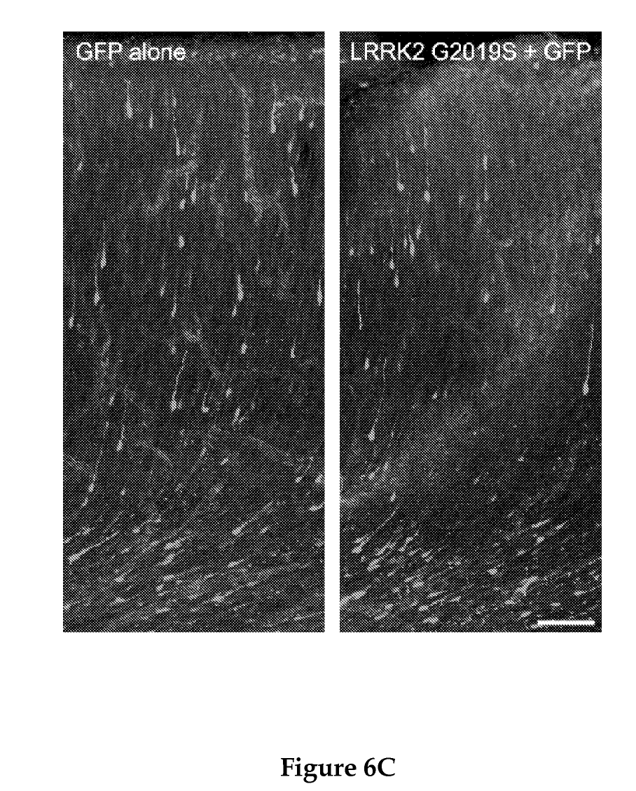

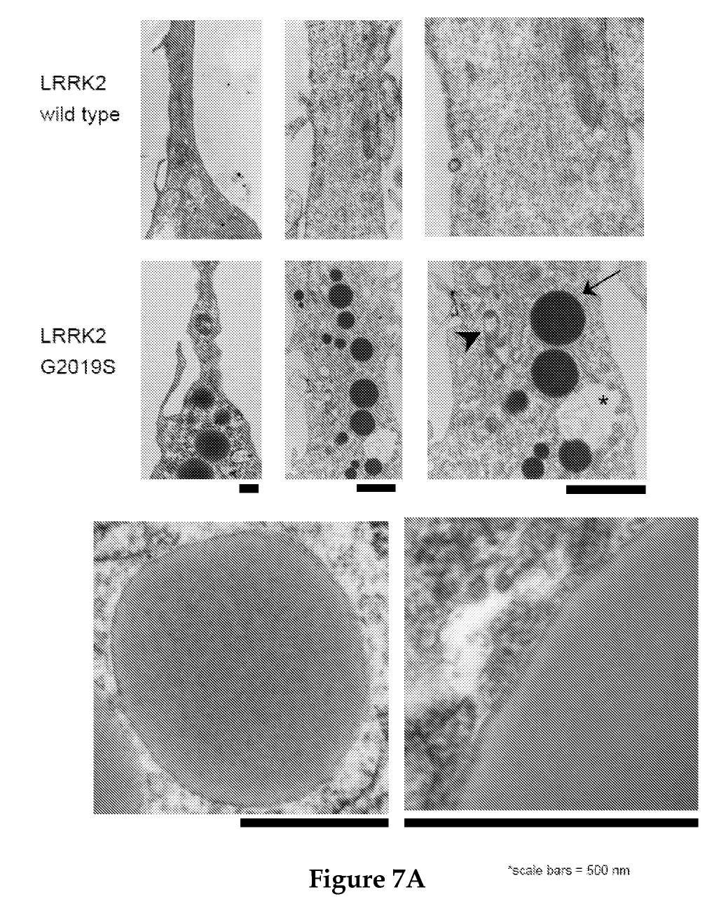

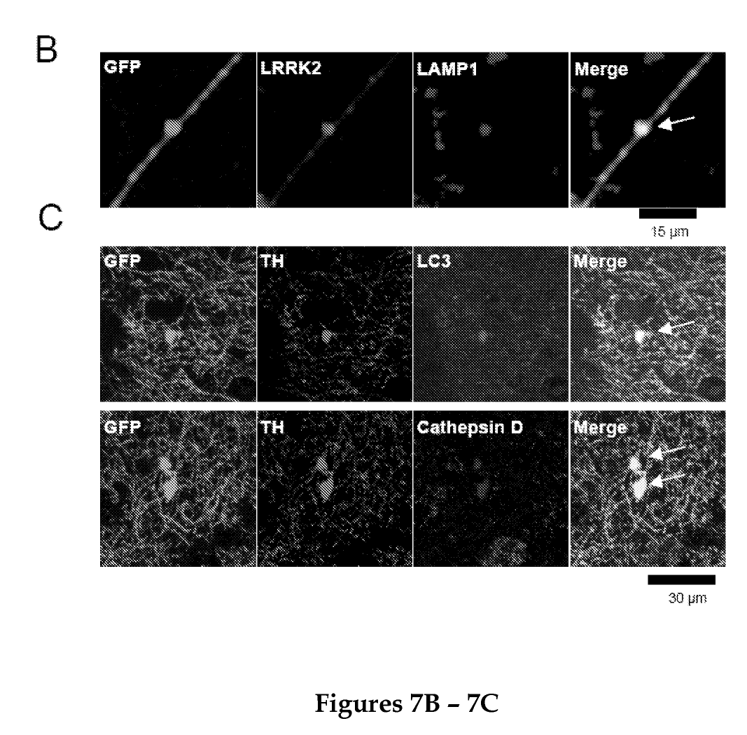

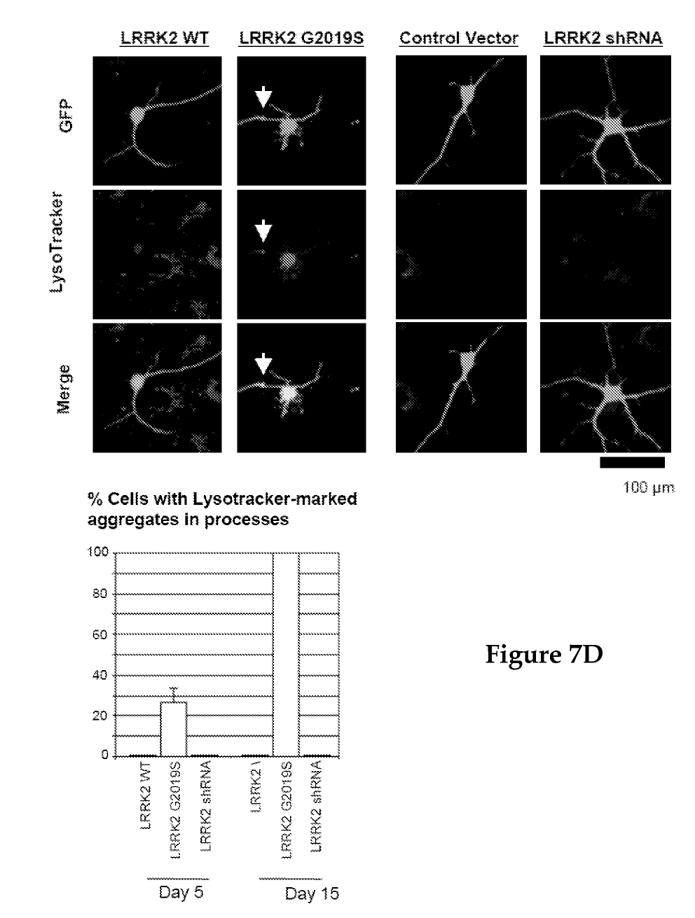

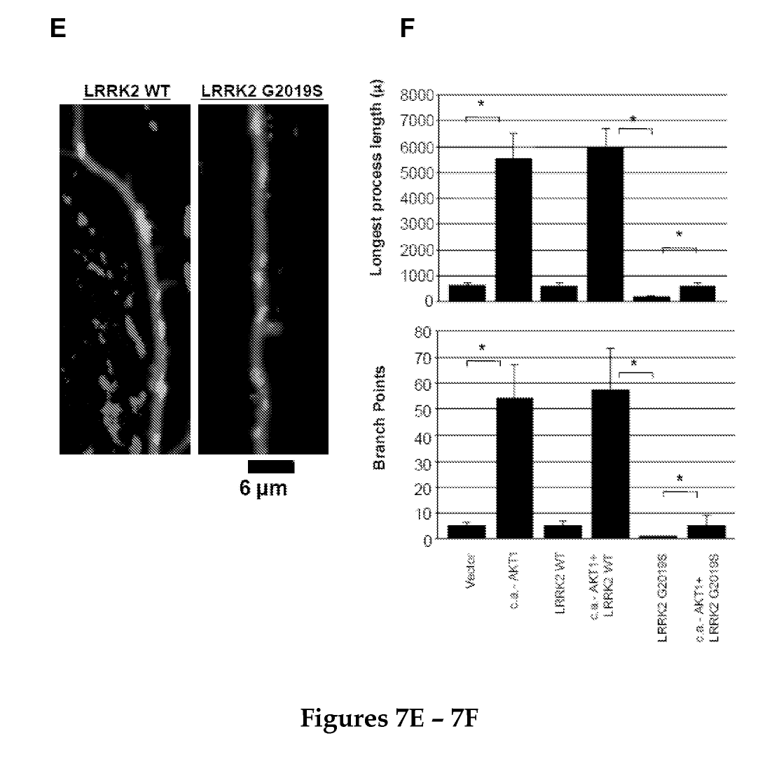

LRRK2 regulates neurite process morphology in the CNS. Axonal processes appeared to be reduced in complexity in the context of G2019S expression relative to vector control in the adult rat gene transduction model (FIGS. 5D and 5C), but this was difficult to quantify due to the high density of neuronal processes in the intact CNS. To circumvent this, the invention provides an animal model in which a technique was used that allows for the marking of individual, genetically altered neurons within an otherwise normal CNS environment: in utero intracerebral gene transduction of rat embryos by vector injection into the lateral ventricles. Genetic manipulation of neuronal progenitors within the periventricular cell layer of E16 rat embryos can be achieved by either plasmid vector electroporation or lentiviral transduction (Tsai et al., 2005). After a 5-day period in utero, the embryos (E21) are sacrificed and brain sections are visualized by confocal fluorescence microscopy. Electroporation of vector alone labels neuronal cells throughout layers 1 and 2 of the cerebral cortex that appear morphologically as neurons and are immunostained with a neuronal marker, TujI (FIGS. 6A and 6C). Overexpression of PD-associated mutant LRRK2 alleles, G2019S or T2020T, reduced both the length and the branching of neuronal processes, relative to control vector (FIGS. 6A and 6B), consistent with the phenotype observed in neuronal primary cultures. Cortical neurons that overexpress wild-type LRRK2 did display a mild reduction in process length, but this was significantly less pronounced than in the PD-associated LRRK2 mutant allele expressing cells. Overexpression of either the mutant or wild-type alleles of LRRK2 significantly reduced the number of branch points in cortical neurons in vivo. Thus, overexpression of the wild-type allele of LRRK2 leads to an altered process phenotype in cortical neurons in the intact CNS, albeit mildly relative to the PD-associated mutants. These data support the notion that an increase in LRRK2 activity leads to a reduction in neuronal process complexity in the intact CNS.

LRRK2 knockdown in cortical neurons in the intact CNS was achieved using lentiviral vectors that harbor either shRNA for LRRK2 or control vector alone along with eGFP marker sequences. Cortical neurons transduced with LRRK2 shRNA display a significant increase in the number of branch points relative to control vector-transduced cells (FIG. 6B). Also, total axon length appeared to be increased in the knockdown cells, although this did not reach statistical significance. In summary, these results show that LRRK2 regulates neuronal process morphology in the intact CNS, consistent with the primary culture analysis.

Cellular mechanism of LRRK2 action. To investigate cellular mechanisms of the Parkinsonism-associated LRRK2 alleles, ultrastructural analyses were performed by electron microscopy. Neurons expressing the LRRK2 G2019S mutant allele, but not control vector, display abnormal accumulation of abundant electrodense structures suggestive of swollen lysosomes (FIG. 7A). Additionally, multivesicular bodies (MVBs), distended mitochondria associated with vacuoles, and disrupted cytoskeletal structures are observed. Consistent with these findings, immunohistochemical analysis and confocal microscopy of neurons in primary culture or in the intact CNS expressing the G2019S LRRK2 allele revealed prominent membranous structures that stain with antibodies for the lysosomal markers LAMP1 (FIG. 7B) and Cathepsin D as well as the autophagosome and lysosome marker LC3 (FIG. 7C). Staining of inclusions was not observed with the early endosome marker EEA1, and uptake of lipophilic dye FM4-86 through early endosomes appeared to be unaltered in the G2019S allele expressing neurons (FIGS. 7H and 7I), showing that early endosome function is intact. Antibody staining for LRRK2 co-localized with the LAMP1 staining at inclusions (FIG. 7B), consistent with biochemical evidence for membrane association of LRRK2 (Gloeckner et al., 2006). A time course analysis using the acidic organelle-specific dye, Lysotracker, that stains lysosomes and late endosomes, demonstrated accumulation as early as 5 days after introduction of G2019S mutant LRRK2 (FIG. 7D). Overexpression of wild-type LRRK2 led to a far less dramatic (but still significant) increase in abnormal Lysotracker staining (FIG. 7D). No abnormal staining was observed with a mitochondrial dye, Mitotracker, in neurons at an early stage (5 days after transfection; FIG. 7E), although at late stages abnormal Mitotracker staining and mitochondrial pathology was evident (14 days; FIG. 7A).

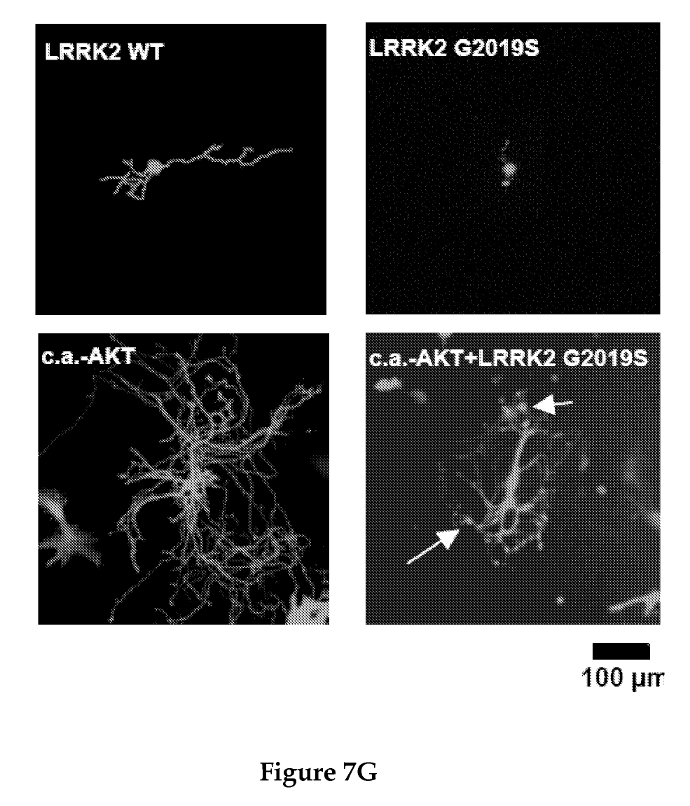

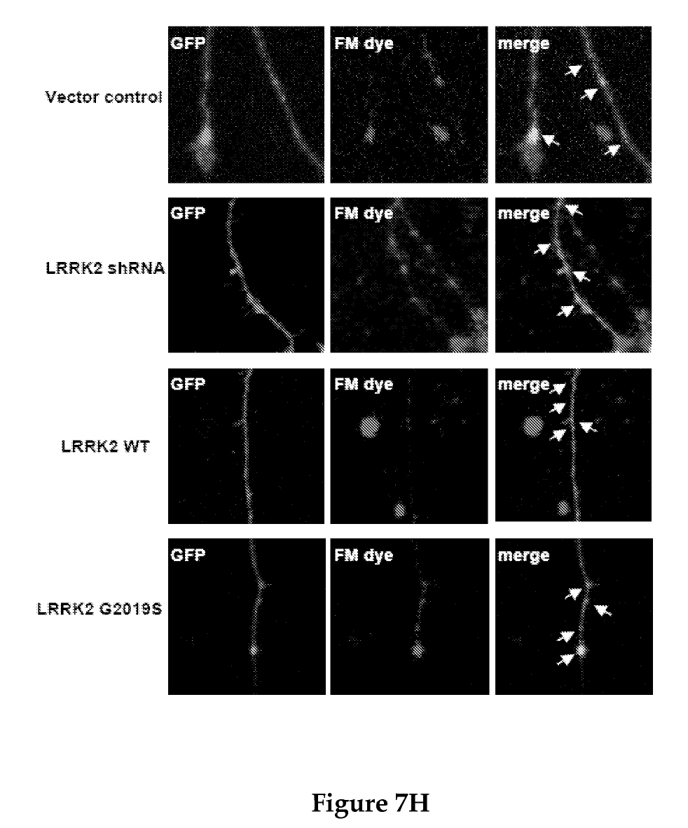

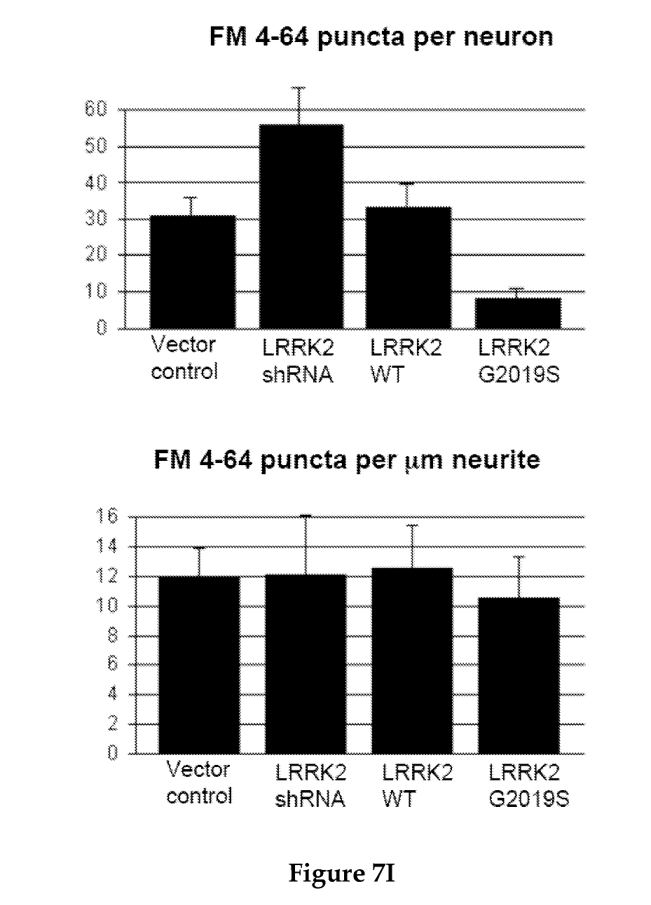

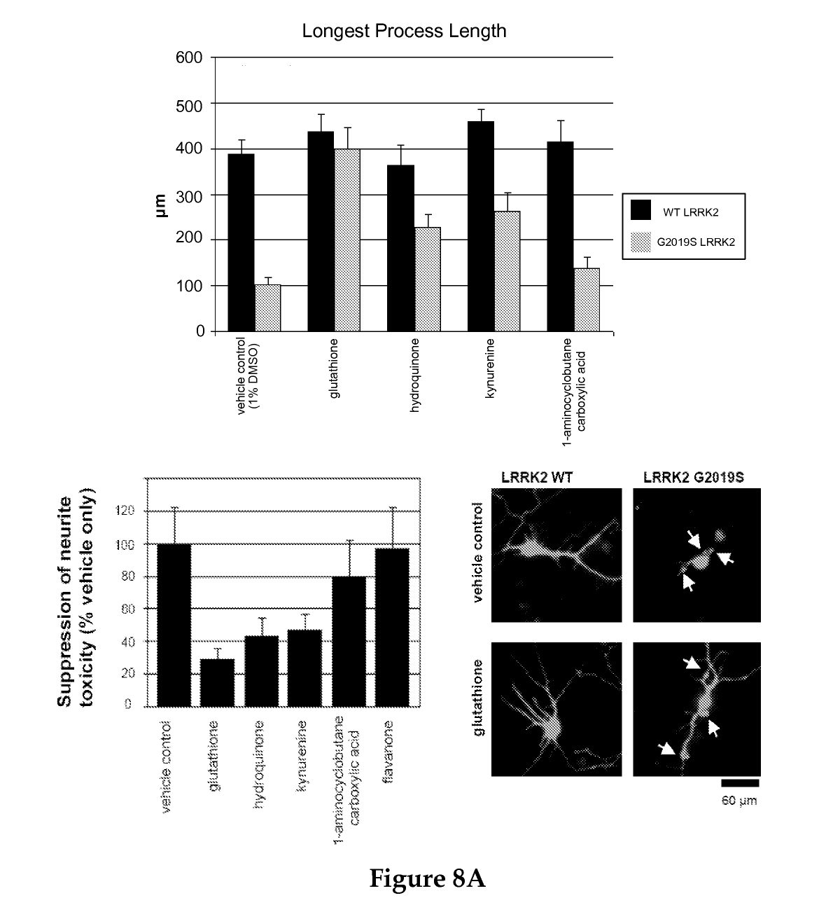

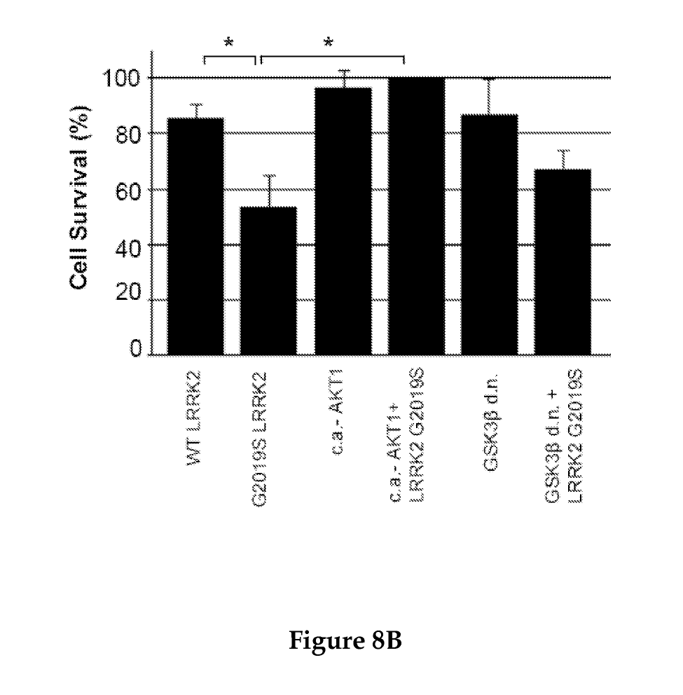

Glutamate excitotoxicity, oxidative stress, and the AKT signaling pathway. The invention provides a chemical genetic approach to probe the molecular signaling mechanism of mutant LRRK2 toxicity in neurons. A library of 1000 diverse and annotated compounds (10 μM) was screened for agents that inhibit the neurite length phenotype of LRRK2 G2019S mutant expression. 30 compounds were identified in an initial screen, and these were then retested for their ability to specifically suppress the G2019S LRRK2 phenotype, as compared to their action on wild-type LRRK2-transfected or vector control-transfected neurons. A total of four agents were identified in this screen that significantly suppressed the neurite length phenotype. Three of these are NMDA glutamate receptor antagonists or partial agonists (kynurenine, 1-aminocyclobutane carboxylic acid (ACBC), and hydroquinone), and two are characterized antioxidants (glutathione and hydroquinone; FIG. 8A). An additional antioxidant, flavanone, displayed mild suppression of the phenotype that did not reach statistical significance. These findings support a role for glutamate excitotoxicity and oxidative stress in LRRK2-mediated cellular phenotypes. Intracellular inclusions and lysosomal changes did not appear to be inhibited by these antioxidant and glutamate antagonists (FIG. 8A), showing that the excitotoxicity and oxidative stress may act downstream of the observed membrane abnormalities. Alternatively, these may be independent phenotypic consequences of LRRK2 mutant allele expression. Given the known role of the AKT kinase signaling pathway in neuron survival in the context of glutamate excitotoxicity and oxidative stress (Datta et al., 1999), as well as the central role of this pathway in the regulation of neurite length and complexity (Shi et al., 2003), experiments were designed to test whether the AKT signaling pathway interacts with LRRK2 induction. Consistent with this model, overexpression of a constitutively active form of AKT1 (Datta et al., 1997) antagonizes the toxicity of G2019S LRRK2 in co-transfection assays in terms of both neuronal morphology and survival (FIGS. 8B, 7F and 7G). In contrast, the spheroid aggregate phenotype does not appear to be rescued by AKT1 activity (FIG. 7G). Similar results were observed by co-transfection of a dominant negative form of GSK-3β, a downstream target of AKT that is inhibited by AKT activation (FIG. 8B).

Pathological analysis of LRRK2-associated Parkinsonism has revealed surprising diversity, including some patients with typical PD pathology (LBs in the SN and loss of midbrain dopamine neurons), and others with broader CNS pathology characteristic of Lewy body disease (α-Synucleinopathy) or progressive supranuclear palsy (α-Taupathy) (Giasson et al., 2006; Wszolek et al., 2004). This diversity implicates LRRK2 broadly in a general cellular mechanism of neurodegeneration. Kinetic analysis of LRRK2 function in primary neuron cultures and in the intact CNS demonstrates a cell-autonomous activity leading initially to prominent lysosomal and mitochondrial membrane perturbations and compromised neurite processes, and subsequently to glutamate excitotoxicity, oxidative stress, and apoptosis. Taken together with the phenotype of cells deficient in LRRK2 activity, which display enhanced neuronal process morphology, these data show a role for LRRK2 in membrane trafficking in neurons. This is consistent with the localization of LRRK2 to these intracellular membrane compartments. Furthermore, a role for LRRK2 in the context of such a basic neuronal cellular function would offer a potential explanation for the diverse neurodegenerative phenotypes associated with clinical mutations in this gene.

Acidic organelle inclusions are an early neuronal phenotype associated with mutant LRRK2 expression. Pathological analyses have previously implicated lysosomal defects and activation in neurodegenerative disorders, particularly in the context of Alzheimer's disease (Cataldo et al., 1994). Genetic studies have also implicated lysosomal alterations in PD, as patients with mutations in glucocerebrosidase harbor lysosomes engorged with stored glycolipid and display a significantly increased incidence of PD (Sidransky, 2004). Furthermore, there is evidence that α-Syn mutations associated with PD leads to lysosomal defects, and some animal models of α-Syn overexpression display lysosomal defects and altered axonal processes that bear similarity to observations in LRRK2 expressing cells (Masliah et al., 2000). Based on kinetic analysis of early events in mutant LRRK2 expressing neurons, it would be of interest to further probe familial Parkinsonism and PD pathological specimens for evidence of early acidic membrane defects.

LRRK2 and the regulation of neurite process maintenance. LRRK2 Parkinsonism-associated mutations lead to defective neurite processes, whereas a reduction in LRRK2 activity leads to exaggerated neuritic processes. Based on these findings, the apparent role of LRRK2 in regulating neurite process maintenance relates directly to altered vesicular membrane trafficking, as prior genetic studies have linked alterations in acidic organelle trafficking with neurite outgrowth changes. For instance, targeted deletion of numb in Drosophila sensory neurons leads to reduced axon length and branching, whereas overexpression leads to exaggerated axons and abnormal lysosomal vesicles (Huang et al., 2005). Furthermore, numb localizes to discrete vesicular structures in neurons. A Drosophila mutation in spinster, a late endosome/lysosome protein, leads to synaptic overgrowth in the context of the accumulation of acidic organelles (Dermaut et al., 2005; Sweeney and Davis, 2002). It remains possible that the mutant LRRK2-associated neurite changes and the acidic organelle defects are independent. Mutations in two other genes linked to autosomal dominant forms of Parkinsonism, α-Synuclein (Masliah et al., 2000) and Tau (Lee et al., 2001; Martin et al., 2001), are also implicated in neurite morphology defects.

Glutamate excitotoxicity and oxidative stress in the context of mutant LRRK2 allele expression. This Example presents evidence that Parkinsonism-associated mutant LRRK2 alleles ultimately lead to glutamate excitotoxicity and oxidative stress. NMDA receptors antagonists and antioxidants suppress the loss of neurites, but not the formation of inclusions or the membrane abnormalities, showing that the glutamate excitotoxicity and oxidative stress are secondary to the membrane trafficking abnormalities. Both glutamate excitotoxicity and oxidative stress have long been implicated in PD (Beal, 2003). Furthermore, these two mechanisms of toxicity are known to function cooperatively at neuron processes; for instance, oxidative stress sensitizes neurons to glutamate excitotoxicity, possibly as a consequence of mitochondrial injury and reduced calcium capacitance (Nicholls et al., 1999). Prominent mitochondrial pathology is observed in ultrastructural analyses of neurons that express Parkinsonism-associated LRRK2. Several prior studies have linked mutations in familial Parkinsonism genes, including PINK1, DJ-1, and Parkin, with altered sensitivity to oxidative stress (Martinat et al., 2004) and mitochondrial dysfunction in neurons (Shen and Cookson, 2004). It will be of interest to investigate possible interactions between these genes and LRRK2, as has been suggested for Parkin (Smith et al., 2005).

Both excitotoxicity and oxidative stress may be secondary consequences of the observed cell membrane changes, based on kinetic analyses, and as prior studies have linked lysosomal dysfunction to secondary mitochondrial defects and oxidative stress (Terman et al., 2006). Alternatively, it is possible that the excitotoxicity and oxidative stress are independent of the cellular pathological membrane changes, or that they function upstream of, and induce, the acidic organelle defects (Butler and Bahr, 2006). Downstream of the excitotoxic insult and NMDA receptor activation, it has been shown that the AKT/GSK3β pathway plays an important role in survival (Datta et al., 1999). Consistent with this, activation of AKT or inhibition of GSK3β both suppress G2019S-mediated toxicity (but do not inhibit inclusion formation).

Potential mechanisms of Tau phosphorylation. Neurons that overexpress LRRK2 display phospho-Tau positive aggregates, as do some patients that harbor LRRK2 mutations. One potential mechanism for the accumulation of phospho-Tau is as a direct result of lysosomal dysfunction (Takauchi and Miyoshi, 1995), as there is evidence that modified Tau is metabolized in lysosomes (Ikeda et al., 2000). Alternatively, glutamate excitotoxicity is predicted to lead to induction of GSK3, as a consequence of reduced AKT activity, and an important direct target of the GSK-3 kinase is believed to be Tau (Mattson, 2001). It is possible that LRRK2 kinase activity would directly phosphorylate Tau or αSyn protein, but phosphorylation of either protein by LRRK2 was not detected in vitro, nor was evidence found for direct physical association.

Regulation of LRRK2 kinase activity. Structure-function analyses define the kinase domain as necessary and sufficient for LRRK2 activity. Furthermore, this Example presents evidence from in vitro kinase assays as well as cell-based phenotypic analyses that additional domains of LRRK2, including the Rho-like Roc domain and the COR domain, function in part to inhibit the kinase activity of LRRK2. Two categories of mutations may lead to LRRK2-associated Parkinsonism: mutations within the kinase domain that lead to disinhibition, such as the G2019S and the I2020T within the activation loop domain; and inactivating mutations throughout the inhibitory domains of LRRK2. Consistent with this, evidence was found for disinhibition of kinase activity in the context of a Roc domain mutation, R1441G.

Animal models and potential therapeutics for LRRK2-associated Parkinsonism. LRRK2 G2019S overexpression in rodent adult dopamine neurons leads to loss of nigrostriatal processes, Tau-positive inclusions, and apoptosis, and is thus a useful animal model for early LRRK2-associated disease. Additional studies in non-human primates are now feasible using the AAV2-based vectors provided by the invention. Furthermore, identified compounds that inhibit LRRK2-mediated toxicity in neurons represent potential therapeutic agents for LRRK2-associated disease.

### Example 2

### NMDA Receptor Antagonists

Mutations in LRRK2 underlie an autosomal dominant, inherited form of Parkinson's disease (PD) that mimics all of the clinical features of the common sporadic form of PD. The LRRK2 protein includes putative GTPase, protein kinase, WD40 repeat, and leucine rich repeat (LRR) domains of unknown function. This example shows that PD-associated LRRK2 pathology and the formation of LC3-GFP labeled aggregates in neurites is ameliorated by the NMDA receptor antagonist L-701,324.

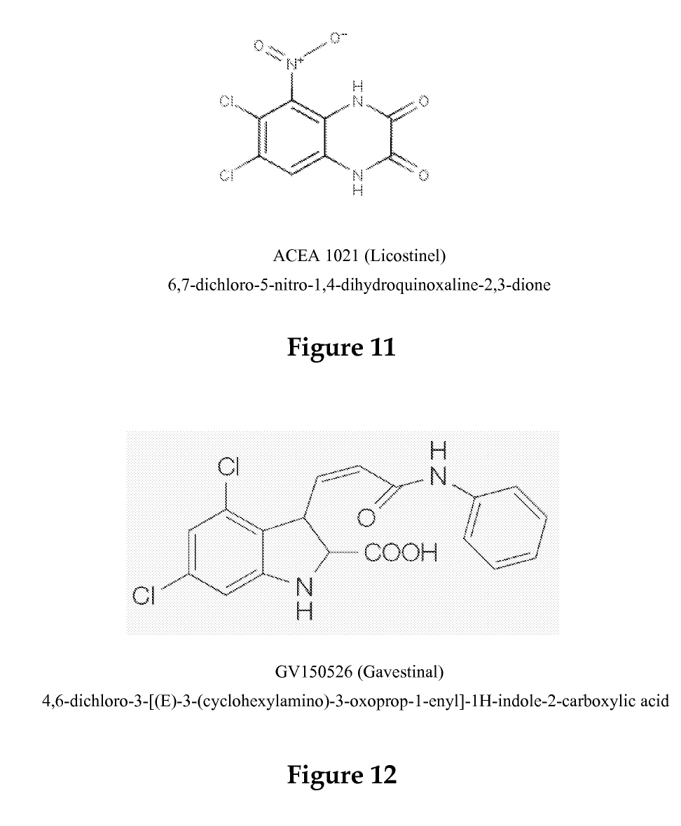

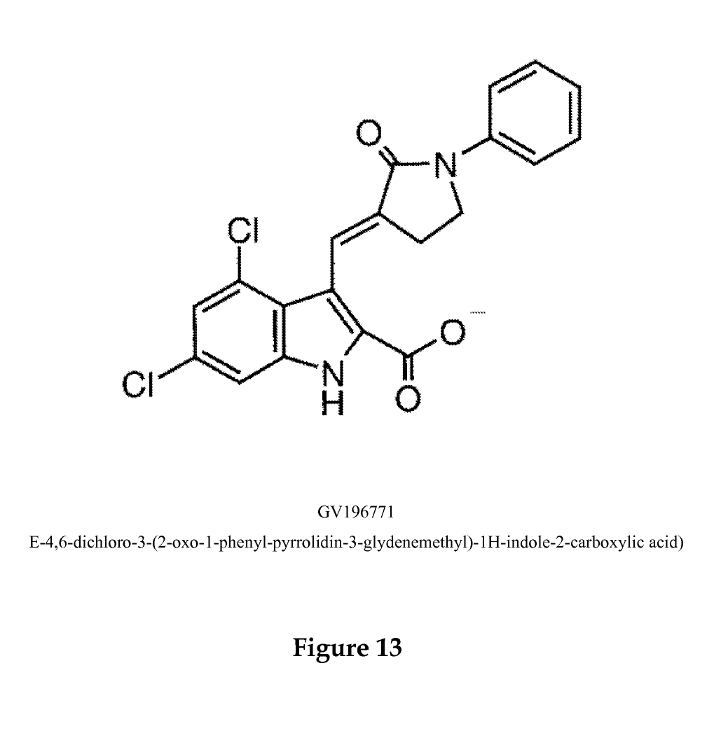

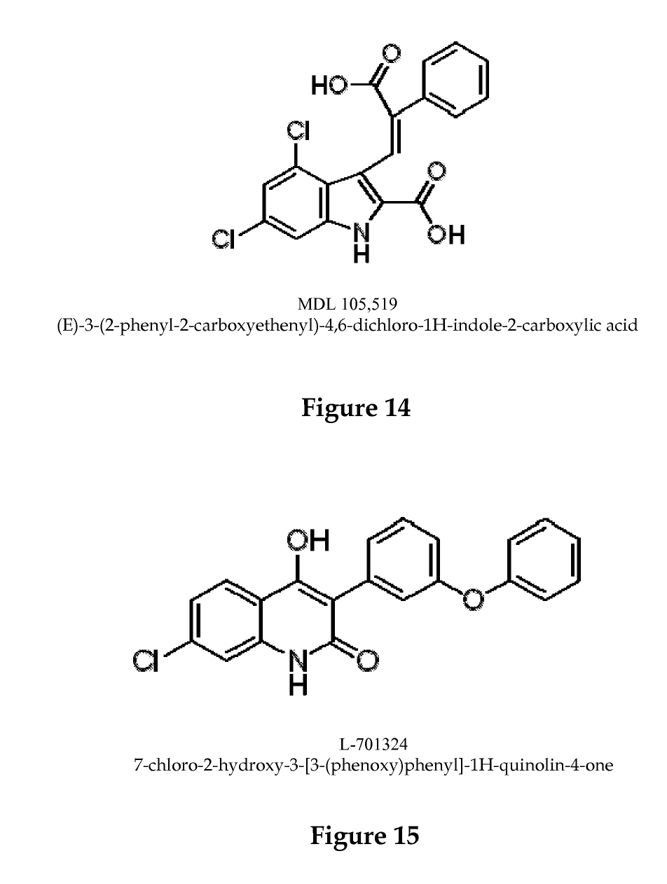

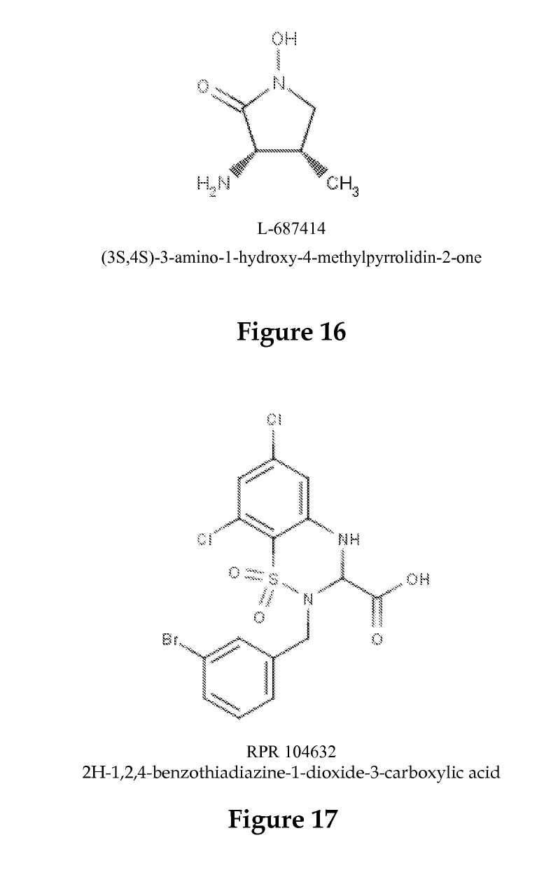

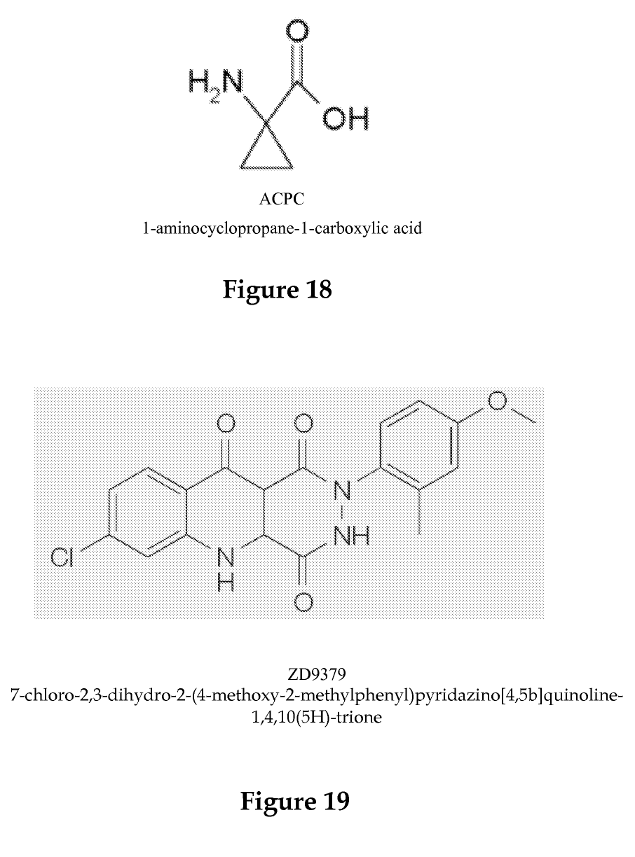

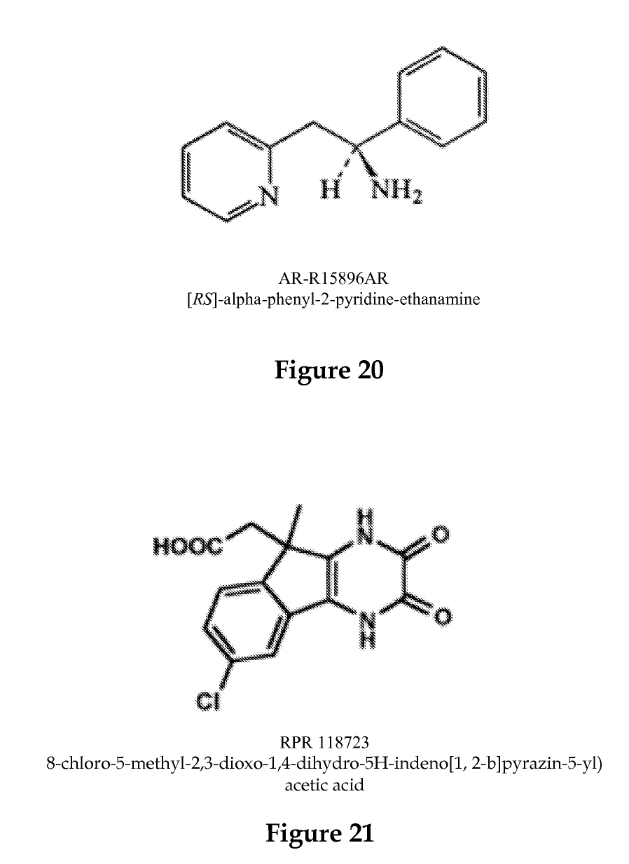

In addition to L-701,324, other compounds are candidate NMDA glycine site antagonists and represent potentially therapeutic molecules for the treatment of neurodegenerative diseases and disorders, such as Parkinson's disease, Alzheimer's disease, and ALS. A non-limiting list of exemplary compounds is provided in Table 1. ACEA 1021, GV150526, GV196711, MDL 105,519, L-701,324, L-687414, RPR 104632, ACPC, ZD9379, and RPR118723 are all glycine site antagonists of the NMDA receptor whereas AR-R15896AR is a low-affinity, use-dependent NMDA antagonist. ACEA 1021 and ACPC represent interesting compounds as potential treatments for PD as neither of these molecules has been previously examined in a neurological context. The chemical structure and IUPAC designations for the compounds listed in Table 1 included in FIGS. 11-21.

TABLE 1
NMDA glycine site antagonists and related agents

| | | Clinical | Route of | | Potential |
|---|---|---|---|---|---|
| Compound | Company | Trial | Administration | Target | Indications |
| ACEA 1021 | Purdue | Phase 3 | Intravenous | NMDA | NSPPE |
| (Licostinel) | | | (IV) | Receptor | (Neurodegener- |
| | | | | Glycine | ation, stroke, pain, |
| | | | | Site (GS) | psychiatric, |
| | | | | | epilepsy) |
| GV150526 | GSK | Phase 3 | IV | GS | NSPPE |
| (Gavestinal) | | | | | |
| GV196771 | GSK | Phase 1/2 | IV, oral (PO) | GS | NSPPE |
| MDL 105,519 | Sanofi Aventis | None | IV | GS | NSPPE |
| L-701324 | Merck | None | IV, PO | GS | NSPPE |
| L-687414 | Merck | Phase 1 | IV | GS | NSPPE |
| RPR | Sanofi Aventis | None | IV | GS | NSPPE |
| 104632 | | | | | |
| ACPC | Transgenomics, | Phase 1 | IV | GS | NSPPE |
| (SYM2030) | Inc. (Annovis, | | | | |
| | Symphony, | | | | |
| | Message) | | | | |
| ZD9379 | Astra | None | IV | GS | NSPPE |
| | Zeneca | | | | |
| AR-R15896AR | Astra | Phase 2 | IV | Comp | NSPPE |
| | Zeneca | | | NMDA | |
| RPR118723 | Sanofi-Aventis | None | IV | GS | NSPPE |

### Example 3

### Experimental Methods

The following methods are non-limiting exemplary methods that may be used in connection with the embodiments of the invention.

Vectors and Cloning. Chimeric LRRK2 cDNA constructs were generated that harbor mouse LRRK2 (SEQ ID NO:3) sequences at the 5′ terminus (GenBank Accession No. NM_{—}025730; bp 1-3738 of the coding sequence) and human LRRK2 (SEQ ID NO:2) sequences downstream (GenBank Accession No. AY_{—}792511; bp 3734-7584 of the coding sequence) and therefore would not be subject to shRNA silencing by a rodent-specific vector. The knockdown shRNA vector targets a region of the rodent LRRK2 gene (SEQ ID NO:3) (GenBank Accession No. NM_{—}025730; bases 4789-4809) that is conserved in rodents but divergent in human LRRK2 cDNA. Total cellular RNA was isolated from mouse midbrain using a Stratagene Absolutely RNA RT-PCR Miniprep Kit and reverse transcribed using an Invitrogen SuperScript First-Strand Synthesis System. The reaction mixture was used directly for PCR with primers specific for mouse LRRK2 (GenBank Accession No. NM_{—}025730); forward primer (nucleotides 1-21) 5′-CAC CTC TGC GGC CGC CAT GGC CAG TGG CGC CTG TCA G-3′ (SEQ ID NO:4), reverse primer (nucleotides 3715-3744) 5′-CTC TAC TCT AGA CCA CAC GTG TGG GTT CTC-3′ (SEQ ID NO:5). The PCR product was inserted into the pENTR/TEV/D-TOPO vector using the MultiSite Gateway Technology recombination system (Invitrogen). Human LRRK2 cDNA from clone DKFZp451G151 (GenBank Accession No. AL832453) (RZPD) (SEQ ID NO:6) was inserted into the pENTR/TEV/D-TOPO vector. Mouse and human cDNA sequences were ligated to generate a 7584 base pair sequence (Mouse: nucleotides 1-3738 of SEQ ID NO:3; Human: nucleotides 3734-7584 of SEQ ID NO:2) and inserted into the pcDNA3.1/nV5 DEST vector using the MultiSite Gateway Technology homologous recombination system (Invitrogen). G2019S, 12020T, K1906M, Y1699C, and R1441G LRRK2 were generated by PCR-mediated mutagenesis.

Immunoprecipitation and Western blot analysis. COS7, HEK293-T or C6 rat glioma cells were maintained in Dulbecco's modified Eagle's medium supplemented with 10% FBS and penicillin/streptomycin. Cells were transfected using Lipofectamine Plus (Life Technologies) and harvested 48 hours after transfection. Transiently transfected COS7 cells were lysed in lysis buffer (10 mM HEPES [pH 7.4], 200 mM NaCl, 100 mM KCl, 1 mM EDTA, 0.5% NP40, 1 mM DTT, 1 mM, NaVO₄, 50 mM NaF and complete protease inhibitors (Sigma)) for 1 h at 4° C. The lysates were cleared by centrifugation at 10,000×g for 15 minutes at 4° C. Protein concentration was determined by the BCA assay (Pierce). For immunoprecipitation, 1.0 mg of lysate was incubated with anti-V5 agarose immobilized antibody overnight at 4° C. The beads were then washed 4 times with lysis buffer, and immunoprecipitated proteins were subjected to in vitro kinase assays. Western blotting was carried out using standard techniques.

In vitro kinase assay. For analysis of LRRK2 kinase activity towards substrates, 293T cells were lysed and immunoprecipitated as described. Immune complexes were incubated in kinase buffer (30 mM Tris[pH 7.5], 20 mM MgCl₂, 2 mM MnCl₂) in the presence of 10 μM [Y−³²P]ATP, 10 mM cold ATP and 1 μg myelin basic protein or myosin light chain protein (Sigma). Phosphorylated substrate was detected by SDS-PAGE and autoradiography.

Cell Culture and Transfection. Sprague-Dawley P1 rat primary dissociated cortical cultures were prepared as previously described (Xia et al., 1996) with modified culture media explained below. Cells were plated at high density, approximately 400,000 cells/cm2, in 24-well plates with 500 μl medium/well. Culture medium used for plating cells was Neurobasal-A supplemented with 2% B-27 and 10% FBS. At 1 day after plating, medium was changed to reduced serum (1% FBS+added antimitotic agents: 70 M uridine and 25 M 5-fluorodeoxyuridine) and replaced weekly thereafter. Cells were transfected at 7 days in vitro using the calcium-phosphate method described (Xia et al., 1996) with the following modifications: no DMSO was added to the transfection mixture, cells were not subjected to glycerol shock, and a total of 3 μg plasmid DNA was used per well.

Immunofluorescence and Microscopy. Cells were fixed in either 2% PFA (for phospho-Tau and tetra-His staining) or 4% PFA for 15 minutes. Fixated cells were treated for 1 hour with blocking solution (10% NDS, 0.1% Triton X-100 in PBS). The following antibodies and dilutions in staining solution (1% NDS, 0.1% Triton X-100 in PBS) were then used for primary immunostaining: mouse anti phospho-Tau AT8 clone (Fitzgerald Industries), 1:100; mouse anti-Tau1 (Chemicon MAB361), 1:200; rabbit anti-cleaved caspase 3 (Cell Signaling), 1:500; mouse anti-α-Synuclein (Transduction Labs) 1:200; anti-Tetra-His (Qiagen), 1:500. Primary staining incubation times were 2 hrs for phospho-Tau and His staining, and otherwise overnight. LAMP1 rabbit polyclonal antibody (GeneTex), 1:100; LRRK2 rabbit polyclonal antibody (Chemicon), 1:200; Mouse monoclonal anti Tau-1 (Chemicon), 1:100; rabbit polyclonal anti Tau phosphoserine-404 (Santa Cruz), 1:200; rabbit polyclonal anti-VMAT2 (Chemicon), 1:200; rabbit polyclonal anti-cleaved Caspase-3 (Cell Signaling), 1:250. Cells were washed 3 times for 10 min in phosphate buffered saline prior to secondary staining. Secondary staining was performed with fluorophoreconjugated (either Cy3 or Cy5) mouse or rabbit anti-IgG antibodies diluted 1:1500 in staining solution for 1 h. Photographs were taken using a Zeiss LSM 510 Meta confocal microscope with excitation and emission filters suitable for eGFP, Cy3, and Cy5 fluorescence. LysoTracker Red DND-99 and MitoTracker Red CMXRos (Invitrogen) were used in culture medium at concentrations of 100 nM and 500 nM, respectively, for live imaging of cells on a Zeiss LSM510 Meta Confocal Microscope.

Electron Microscopy. Performed as in Troy et al. (Troy et al., 1992). Briefly, cells were fixed overnight in 2% glutaraldehyde 1% PFA in PBS, and then in 1% osmium tetroxide (Electron Microscopy Sciences, Ft. Wash., Pa.) for 20 min, then dehydrated in pure ethanol and infiltrated overnight with Epon 812 (SPI Supplies, West Chester, Pa.). Epon was then polymerized at 60° C. for 24 h, cooled and embedded in a larger Epon capsule. Sections (60-90 nm) were cut with an MT5000 ultramicrotome, stained with uranyl acetate and lead citrate. Images were taken with a JEOL 100S Electron Microscope (JEOL USA, Crawford, N.J.).

Annotated Compound Screen. The Spectrum Collection library (MicroSource Discovery Systems) was screened using the cell assay as above in 96-well format. Drug stocks were diluted 1:1000 in cell culture medium. Cells were treated every 48 hours after transfection.

Quantitative Analysis. Images were analyzed using Image-Pro Plus (Mediacybernetics) software version 5.1.0.20. Parameters measured were: the length of the longest neuronal process, the total length of all neuronal processes, and the diameter of the soma along its longest axis. Branch points in the longest process were counted. Statistical analysis was performed using Statview software version 5.0. P values were obtained using Fisher's post-hoc ANOVA.

In utero gene transduction. E16.5 rat embryos were injected in utero into the lateral ventricles with lentiviral vectors as specified. Alternatively, embryos were injected with 1 μl of DNA plasmid, as specified, at a concentration of 1 μg/μl, and panel electroporated across the uterine wall at 50V (pulse length 50 ms) using an Electrosquareporator (BTX Inc.) (Tsai et al., 2005). Embryos were harvested at E20.5, fixed in 4% PFA, and after 24 hours embedded in agarose, and fixed for a further 3 days. The cortices were vibratome sectioned (60 μm thick) and GFP positive cell were imaged using an LSM 510 Meta confocal microscope (Zeiss). Images were then analyzed (by an observer blind to the genotype) with regard to the length of the longest process and the number of branch points off the longest process using Image Pro Plus software and the resulting data was statistically analyzed by ANOVA.

In vivo viral transduction. Anesthetized 5-week old rats were intracranially injected with AAV2 virus expressing IRES-GFP (Stratagene) and AAV2 virus expressing HR-GFP (Stratagene) in one hemisphere. The opposing hemisphere was injected with AAV2 expressing LRRK2 wild-type or G2019S mutated kinase domain:IRESGFP and AAV2 virus expressing HR-GFP. Injections were targeted to the substantia nigra (co-ordinates: AP −5.2; ML ±2.1; DV −7.7). Rats were sacrificed 20 days after injection, subject to intracardiac perfusion with 4% paraformaldehyde, sectioned and immunohistochemically stained, with sheep α-TH (Pelfreeze; 1:500), mouse α-tau-1 (Chemicon; 1:200), rabbit α-P-tau (Santa Cruz; 1:200), rabbit α-VMAT2 (Chemicon; 1:200), rabbit α-activated caspase-3 (Cell Signaling; 1:200), rabbit α-cathepsin D (DAKO; 1:200) or rabbit α-LC3 (Santa Cruz; 1:200) and appropriate secondary antibodies from Jackson Laboratories (1:500). Sections from animals with similar levels of GFP labeling in the substantia nigra were then imaged using a Zeiss LSM510 confocal microscope at the levels of the substantia nigra and the striatum.

### REFERENCES

Abeliovich, A., and Beal, M. F. (2006). Parkinsonism genes: culprits and clues. J Neurochem. 99, 1062-72.
Beal, M. F. (2003). Mitochondria, oxidative damage, and inflammation in Parkinson's disease. Ann N Y Acad Sci 991, 120-131.
Bosgraaf, L., and Van Haastert, P. J. (2003). Roc, a Ras/GTPase domain in complex proteins. Biochim Biophys Acta 1643, 5-10.
Butler, D., and Bahr, B. A. (2006). Oxidative stress and lysosomes: CNS-related consequences and implications for lysosomal enhancement strategies and induction of autophagy. Antioxid Redox Signal 8, 185-196.
Cataldo, A. M., Hamilton, D. J., and Nixon, R. A. (1994). Lysosomal abnormalities in degenerating neurons link neuronal compromise to senile plaque development in Alzheimer disease. Brain Res 640, 68-80.
Cobb, M. H., and Goldsmith, E. J. (1995). How MAP kinases are regulated. J Biol Chem 270, 14843-14846.
Datta, S. R., Brunet, A., and Greenberg, M. E. (1999). Cellular survival: a play in three Akts. Genes Dev 13, 2905-2927.
Datta, S. R., Dudek, H., Tao, X., Masters, S., Fu, H., Gotoh, Y., and Greenberg, M. E. (1997). Akt phosphorylation of BAD couples survival signals to the cell-intrinsic death machinery. Cell 91, 231-241.
Davies, H., Bignell, G. R., Cox, C., Stephens, P., Edkins, S., Clegg, S., Teague, J., Woffendin, H., Garnett, M. J., Bottomley, W., et al. (2002). Mutations of the BRAF gene in human cancer. Nature 417, 949-954.
Dermaut, B., Norga, K. K., Kania, A., Verstreken, P., Pan, H., Zhou, Y., Callaerts, P., and Bellen, H. J. (2005). Aberrant lysosomal carbohydrate storage accompanies endocytic defects and neurodegeneration in Drosophila benchwarmer. J Cell Biol 170, 127-139.
Di Fonzo, A., Rohe, C. F., Ferreira, J., Chien, H. F., Vacca, L., Stocchi, F., Guedes, L., Fabrizio, E., Manfredi, M., Vanacore, N., et al. (2005). A frequent LRRK2 gene mutation associated with autosomal dominant Parkinson's disease. Lancet 365, 412-415.
Emre, M. (2003). Dementia associated with Parkinson's disease. Lancet Neurol 2, 229-237.
Galvin, J. E., Lee, V. M., and Trojanowski, J. Q. (2001). Synucleinopathies: clinical and pathological implications. Arch Neurol 58, 186-190.
Gasser, T. (2005). Genetics of Parkinson's disease. Curr Opin Neurol 18, 363-369.
Giasson, B. I., Covy, J. P., Bonini, N. M., Hurtig, H. I., Farrer, M. J., Trojanowski, J. Q., and Van Deerlin, V. M. (2006). Biochemical and pathological characterization of Lrrk2. Ann Neurol 59, 315-322.
Gilks, W. P., Abou-Sleiman, P. M., Gandhi, S., Jain, S., Singleton, A., Lees, A. J., Shaw, K., Bhatia, K. P., Bonifati, V., Quinn, N. P., et al. (2005). A common LRRK2 mutation in idiopathic Parkinson's disease. Lancet 365, 415-416.
Gloeckner, C. J., Kinkl, N., Schumacher, A., Braun, R. J., O'Neill, E., Meitinger, T.,
Kolch, W., Prokisch, H., and Ueffing, M. (2006). The Parkinson disease causing LRRK2 mutation I2020T is associated with increased kinase activity. Hum Mol Genet 15, 223-232.
Huang, E. J., Li, H., Tang, A. A., Wiggins, A. K., Neve, R. L., Zhong, W., Jan, L. Y., and Jan, Y. N. (2005). Targeted deletion of numb and numblike in sensory neurons reveals their essential functions in axon arborization. Genes Dev 19, 138-151.
Ikeda, K., Akiyama, H., Arai, T., Kondo, H., Haga, C., Tsuchiya, K., Yamada, S., Murayama, S., and Hori, A. (2000). Neurons containing Alz-50-immunoreactive granules around the cerebral infarction: evidence for the lysosomal degradation of altered tau in human brain? Neurosci Lett 284, 187-189.
Lee, V. M., Goedert, M., and Trojanowski, J. Q. (2001). Neurodegenerative tauopathies. Annu Rev Neurosci 24, 1121-1159.
Lesage, S., Durr, A., Tazir, M., Lohmann, E., Leutenegger, A. L., Janin, S., Pollak, P., and Brice, A. (2006). LRRK2 G2019S as a cause of Parkinson's disease in North African Arabs. N Engl J Med 354, 422-423.
Manning, G., Whyte, D. B., Martinez, R., Hunter, T., and Sudarsanam, S. (2002). The protein kinase complement of the human genome. Science 298, 1912-1934.
Marder, K., Levy, G., Louis, E. D., Mejia-Santana, H., Cote, L., Andrews, H., Harris, J., Waters, C., Ford, B., Frucht, S., et al. (2003). Familial aggregation of early- and late-onset Parkinson's disease. Ann Neurol 54, 507-513.
Martin, E. R., Scott, W. K., Nance, M. A., Watts, R. L., Hubble, J. P., Koller, W. C., Lyons, K., Pahwa, R., Stern, M. B., Colcher, A., et al. (2001). Association of singlenucleotide polymorphisms of the tau gene with late-onset Parkinson disease. Jama 286, 2245-2250.
Martinat, C., Shendelman, S., Jonason, A., Leete, T., Beal, M. F., Yang, L., Floss, T., and Abeliovich, A. (2004). Sensitivity to oxidative stress in DJ-1-deficient dopamine neurons: an ES-derived cell model of primary parkinsonism. PLoS Biol 2, e327.
Masliah, E., Rockenstein, E., Veinbergs, I., Mallory, M., Hashimoto, M., Takeda, A., Sagara, Y., Sisk, A., and Mucke, L. (2000). Dopaminergic loss and inclusion body formation in alpha synuclein mice: implications for neurodegenerative disorders. Science 287, 1265-1269.
Mattson, M. P. (2001). Neuronal death and GSK-3beta: a tau fetish? Trends Neurosci 24, 255-256.
Nicholls, D. G., Budd, S. L., Castilho, R. F., and Ward, M. W. (1999). Glutamate excitotoxicity and neuronal energy metabolism. Ann N Y Acad Sci 893, 1-12.
Nichols, W. C., Pankratz, N., Hernandez, D., Paisan-Ruiz, C., Jain, S., Halter, C. A., Michaels, V. E., Reed, T., Rudolph, A., Shults, C. W., et al. (2005). Genetic screening for a single common LRRK2 mutation in familial Parkinson's disease. Lancet 365, 410-412.
Ozelius, L. J., Senthil, G., Saunders-Pullman, R., Ohmann, E., Deligtisch, A., Tagliati, M., Hunt, A. L., Klein, C., Henick, B., Hailpern, S. M., et al. (2006). LRRK2 G2019S as a cause of Parkinson's disease in Ashkenazi Jews. N Engl J Med 354, 424-425.
Paisan-Ruiz, C., Jain, S., Evans, E. W., Gilks, W. P., Simon, J., van der Brug, M., de Munain, A. L., Aparicio, S., Gil, A. M., Khan, N., et al. (2004). Cloning of the gene containing mutations that cause PARK8-linked Parkinson's disease. Neuron 44, 595-600.
Shen, J., and Cookson, M. R. (2004). Mitochondria and dopamine: new insights into recessive parkinsonism. Neuron 43, 301-304.
Shi, S. H., Jan, L. Y., and Jan, Y. N. (2003). Hippocampal neuronal polarity specified by spatially localized mPar3/mPar6 and PI 3-kinase activity. Cell 112, 63-75.
Sidransky, E. (2004). Gaucher disease: complexity in a “simple” disorder. Mol Genet Metab 83, 6-15.
Smith, W. W., Pei, Z., Jiang, H., Moore, D. J., Liang, Y., West, A. B., Dawson, V. L., Dawson, T. M., and Ross, C. A. (2005). Leucine-rich repeat kinase 2 (LRRK2) interacts with parkin, and mutant LRRK2 induces neuronal degeneration. Proc Natl Acad Sci USA 102, 18676-18681.
Sweeney, S. T., and Davis, G. W. (2002). Unrestricted synaptic growth in spinster-a late endosomal protein implicated in TGF-beta-mediated synaptic growth regulation. Neuron 36, 403-416.
Takauchi, S., and Miyoshi, K. (1995). Cytoskeletal changes in rat cortical neurons induced by long-term intraventricular infusion of leupeptin. Acta Neuropathol (Berl) 89, 8-16.
Terman, A., Gustafsson, B., and Brunk, U. T. (2006). The lysosomal-mitochondrial axis theory of postmitotic aging and cell death. Chem Biol Interact.
Troy, C. M., Greene, L. A., and Shelanski, M. L. (1992). Neurite outgrowth in peripherin-depleted PC12 cells. J Cell Biol 117, 1085-1092.
Tsai, J. W., Chen, Y., Kriegstein, A. R., and Vallee, R. B. (2005). LIS1 RNA interference blocks neural stem cell division, morphogenesis, and motility at multiple stages. J Cell Biol 170, 935-945.
West, A. B., Moore, D. J., Biskup, S., Bugayenko, A., Smith, W. W., Ross, C. A., Dawson, V. L., and Dawson, T. M. (2005). Parkinson's disease-associated mutations in leucine-rich repeat kinase 2 augment kinase activity. Proc Natl Acad Sci USA 102, 16842-16847.
Wszolek, Z. K., Pfeiffer, R. F., Tsuboi, Y., Uitti, R. J., McComb, R. D., Stoessl, A. J., Strongosky, A. J., Zimprich, A., Muller-Myhsok, B., Farrer, M. J., et al. (2004). Autosomal dominant parkinsonism associated with variable synuclein and tau pathology. Neurology 62, 1619-1622.
Xia, Z., Dudek, H., Miranti, C. K., and Greenberg, M. E. (1996). Calcium influx via the NMDA receptor induces immediate early gene transcription by a MAP kinase/ERKdependent mechanism. J Neurosci 16, 5425-5436.
Zecca, L., Zucca, F. A., Wilms, H., and Sulzer, D. (2003). Neuromelanin of the substantia nigra: a neuronal black hole with protective and toxic characteristics. Trends Neurosci 26, 578-580.
Zimprich, A., Biskup, S., Leitner, P., Lichtner, P., Farrer, M., Lincoln, S., Kachergus, J., Hulihan, M., Uitti, R. J., Calne, D. B., et al. (2004). Mutations in LRRK2 cause autosomal-dominant parkinsonism with pleomorphic pathology. Neuron 44, 601-607.

What is claimed is: Methods of treatment

1. A method for treating a neurodegenerative disease in a subject, the method comprising administering to the subject an effective amount of a compound of Formula I: 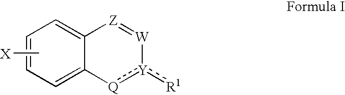
or a pharmaceutically acceptable base or acid addition salt, hydrate, stereoisomer, or mixture thereof, wherein
X is one or more halogen radicals;
Q is NH or N;
W is CR², CHR², NR³, or CH═COH;
Y is C or CH;
Z is C═O, SO₂, COH, CHOH, or NHR⁴;
R¹ is CO₂H or oxo (═O);
R² is H, C(═O)—C₁-C₆ alkyl, C(═O)O—C₁-C₆ alkyl, or C(═O)—C₃-C₈ cycloalkyl;
R³ is optionally substituted C₃-C₁₀ aryl; and
R⁴ is 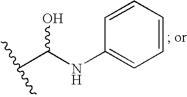
R² and R¹ combine with the carbons to which they are attached to form a 6 membered heterocycle that is optionally substituted at one or more of the heteroatoms with C₃-C₁₀ aryl, wherein the aryl may be substituted with one or more of C₁-C₆ alkyl or —O—C₁-C₆ alkyl, and the heteroatoms in the heterocyclic ring are one or more nitrogen atoms.

2. The method of claim 1, wherein X is one Cl radical at the 7 position of the fused benzene ring.

3. The method of claim 1, wherein X is two Cl radicals at the 5 and 7 positions of the fused benzene ring.

4. The method of claim 1, wherein Z is SO₂.

5. The method of claim 1, wherein Z is C═O.

6. The method of claim 1, wherein Z is COH.

7. The method of claim 1, wherein Z is CHOH.

8. The method of claim 1, wherein Z is NHR⁴, and R⁴ is

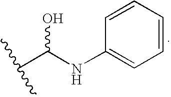

9. The method of claim 1, wherein Q is N, Y is C, and R¹ is CO₂H.

10. The method of claim 1, wherein Q is NH, Y is C, and R¹ is oxo.

11. The method of claim 1, wherein Q is NH, Y is CH, and R¹ is CO₂H.

12. The method of claim 1, wherein W is CHR², and R² is C(═O)O—C₁-C₆ alkyl.

13. The method of claim 1, wherein R² is C(═O)O-methyl.

14. The method of claim 1, wherein W is CHR², and R² is C(═O)—C₃-C₈ cycloalkyl.

15. The method of claim 1, wherein R² is C(═O)-cyclopropyl.

16. The method of claim 1, wherein W is NR³.

17. The method of claim 1, wherein R³ is benzyl.

18. The method of claim 1, wherein R³ is benzyl substituted with a halogen.

19. The method of claim 1, wherein W is NR³ and R³ is meta-bromo-benzyl.

20. A method for treating a neurodegenerative disease in a subject, the method comprising administering to the subject an effective amount of a compound of Formula II: 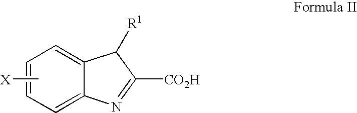
or a pharmaceutically acceptable base or acid addition salt, hydrate, stereoisomer, or mixture thereof, wherein
X is one or more halogen radicals;
R¹ is (CH₂)ₙ—CO₂H, or CH═CHC(═O)NHR²; and
R² is C₃-C₁₀ aryl optionally substituted with one or more of C₁-C₆ alkyl, —O—C₁-C₆ alkyl, or halogen; and
n is 0, 1, 2, 3, 4, 5, or 6.

21. The method of claim 20, wherein X is one Cl radical at the 7 position of the fused benzene ring.

22. The method of claim 20, wherein X is two Cl radicals at the 5 and 7 positions of the fused benzene ring.

23. The method of claim 20, wherein R¹ is CH₂CO₂H.

24. The method of claim 20, wherein R¹ is (CH₂)₂CO₂H.

25. The method of claim 20, wherein R¹ is CH═CHC(═O)NHR², and R² is phenyl.

26. The method of claim 1 or 20, wherein the compound is an antagonist of a NMDA receptor glycine site.

27. A method for treating a neurodegenerative disease in a subject, the method comprising administering to the subject an effective amount of a compound selected from the group consisting of: ACEA 1012 (Licostinel), 5,7-dichlorokynurenic acid, L689,560, L701,252, L687,414, SC49648, MDL29,951, MDL105,519, GV150526 (Gavestinal) GV196771, RPR104,632, RPR118723, L695,902, ZD9379, 2-amino-5-phosphonopentanoate (AP-5), MK-801, L701,324, kynurenine, 1-aminocyclobutane carboxylic acid (ACBC), 1-aminocyclopentane-1-carboxylic acid (ACPC), AR-R15896AR, hydroquinone, and glutathione.

28. The method of claim 1 or 20 wherein the treating comprises preventing the neurodegenerative disease, slowing the onset or progression of the neurodegenerative disease, alleviating one or more symptoms of the neurodegenerative disease, or any combination thereof.

29. The method of claim 1 or 20, wherein the neurodegenerative disease comprises sporadic Parkinson's disease, autosomal recessive early-onset Parkinson's disease, Alzheimer's disease, stroke, amyotrophic lateral sclerosis, Binswanger's disease, Huntington's chorea, multiple sclerosis, myasthenia gravis or Pick's disease.

30. The method of claim 1 or 20, wherein the neurodegenerative disease comprises a mutation in leucine-rich repeat kinase-2 (LRRK2).
Mutant LRRK2 assays

31. A method for determining whether a compound inhibits mutant leucine-rich repeat kinase-2 (LRRK2) protein activity, the method comprising:
(a) expressing in a primary neuronal cell a Parkinson's Disease-associated LRRK2 mutant protein, wherein expression of the mutant results in accumulation of axonal spheroid inclusions in the cell that stain positive for Tau protein;
(b) contacting the neuronal cell with a compound; and
(c) determining whether accumulation of axonal spheroid inclusions in the neuronal cell is reduced compared to accumulation of axonal spheroid inclusions in a neuronal cell expressing the LRRK2 mutant in the absence of the compound; wherein determination of a reduction in (c) indicates that the compound inhibits the LRRK2 mutant protein activity.

32. A method for determining whether a compound inhibits mutant leucine-rich repeat kinase-2 (LRRK2) protein activity, the method comprising:
(a) expressing in a primary neuronal cell a Parkinson's Disease-associated LRRK2 mutant protein, wherein expression of the mutant results in decreased axonal length;
(b) contacting the neuronal cell with a compound; and
(c) determining whether axonal length in the neuronal cell is increased compared to axonal length in a neuronal cell expressing the LRRK2 mutant in the absence of the compound,
wherein determination of an increase in (c) indicates that the compound inhibits the LRRK2 mutant protein activity.

33. A method for determining whether a compound inhibits mutant leucine-rich repeat kinase-2 (LRRK2) protein activity, the method comprising:
(a) expressing in a primary neuronal cell a Parkinson's Disease-associated LRRK2 mutant, wherein expression of the mutant results in decreased axonal branching;
(b) contacting the neuronal cell with a compound; and
(c) determining whether axonal branching in the neuronal cell is increased compared to axonal branching in a neuronal cell expressing the LRRK2 mutant in the absence of the compound,
wherein determination of an increase in (c) indicates that the compound inhibits the LRRK2 mutant protein activity.

34. The method of claim 31, wherein the primary neuronal cell comprises a nucleic acid vector encoding a Parkinson's Disease-associated LRRK2 mutant protein.

35. The method of claim 31, wherein the LRRK2 mutant protein consists essentially of a LRRK2 kinase domain, wherein the kinase domain comprises one or more Parkinson's Disease-associated LRRK2 mutations.

36. The method of claim 31, wherein the LRRK2 mutant protein comprises a G2019S mutation, a I2020T mutation, or both.

37. The method of claim 31, wherein the vector is a lentiviral vector, an adeno-associated virus-2 (AAV-2) vector, an adenoviral vector, a retroviral vector, a polio viral vector, a murine Maloney-based viral vector, an alpha viral vector, a pox viral vector, a herpes viral vector, a vaccinia viral vector, a baculoviral vector, or a parvoviral vector.

38. The method of claim 31, wherein the primary neuronal cell is in vivo in an animal.

39. The method of claim 31, wherein the primary neuronal cell is in a cell culture.

40. The method of claim 31, wherein the primary neuronal cell is a post-mitotic neuron.

41. The method of claim 31, wherein the post-mitotic neuron is a cortical neuron, a dopamine neuron, or a sympathetic neuron.

42. The method of claim 31, further comprising expressing a fluorescent protein in the primary neuronal cell.

43. The method of claim 31, wherein the determining comprises detecting fluorescence.

44. The method of claim 31, wherein the determining comprises computer-assisted quantification of axonal length.

45. The method of claim 31, wherein the determining comprises computer-assisted quantification of axonal branching.

46. The method of claim 31, wherein the compound comprises a peptide fragment of a LRRK2 protein.

47. The method of claim 31, wherein the compound consists essentially of a LRRK2 kinase domain.

48. The method of claim 31, wherein the compound comprises a nucleic acid, or a polypeptide expressed therefrom, capable of inhibiting expression of a LRRK2 protein.

49. The method of claim 31, wherein the nucleic acid comprises RNA, antisense RNA, small interfering RNA (siRNA), double stranded RNA (dsRNA), short hairpin RNA (shRNA), cDNA, DNA, or any combination thereof.

50. The method of claim 31, wherein the compound is a N-methyl-D-aspartic acid (NMDA) receptor antagonist.

51. The method of claim 31, wherein the NMDA receptor antagonist is a NMDA glycine site antagonist.

52. The method of claim 31, wherein the compound is a compound of Formula I: 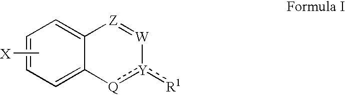
or a pharmaceutically acceptable base or acid addition salt, hydrate, stereoisomer, or mixture thereof, wherein
X is one or more halogen radicals;
Q is NH or N;
W is CR², CHR², NR³, or CH═COH;
Y is C or CH;
Z is C═O, SO₂, COH, CHOH, or NHR⁴;
R¹ is CO₂H or oxo (═O);
R² is H, C(═O)—C₁-C₆ alkyl, C(═O)O—C₁-C₆ alkyl, or C(═O)—C₃-C₈ cycloalkyl;
R³ is optionally substituted C₃-C₁₀ aryl; and
R⁴ is 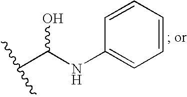
R² and R¹ combine with the carbons to which they are attached to form a 6 membered heterocycle that is optionally substituted at one or more of the heteroatoms with C₃-C₁₀ aryl, wherein the aryl may be substituted with one or more of C₁-C₆ alkyl or —O—C₁-C₆ alkyl, and the heteroatoms in the heterocyclic ring are one or more nitrogen atoms.

53. The method of claim 31, where in the compound is a compound of Formula II: 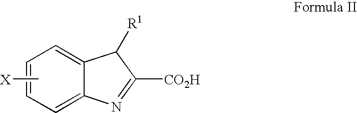
or a pharmaceutically acceptable base or acid addition salt, hydrate, stereoisomer, or mixture thereof, wherein
X is one or more halogen radicals;
R¹ is (CH₂)ₙ—CO₂H, or CH═CHC(═O)NHR²; and
R² is C₃-C₁₀ aryl optionally substituted with one or more of C₁-C₆ alkyl, —O—C₁-C₆ alkyl, or halogen; and
n is 0, 1, 2, 3, 4, 5, or 6.

54. The method of claim 31, wherein the method is carried out in a multi-well plate.

55. The method of claim 31, wherein the method is carried out in a high-throughput manner.

56. The method of claim 31, wherein the method is carried out for more than one hundred compounds.

57. A method for inhibiting activity of a Parkinson's disease-associated LRRK2 mutant protein in a neuronal cell, the method comprising contacting the cell with an N-methyl-D-aspartic acid (NMDA) receptor antagonist.

58. A method for inhibiting activity of a Parkinson's disease-associated LRRK2 mutant protein in a neuronal cell, the method comprising contacting the cell with an antioxidant.

59. A method for inhibiting activity of a Parkinson's disease-associated LRRK2 mutant protein in a neuronal cell, the method comprising contacting the cell with a compound of Formula I: 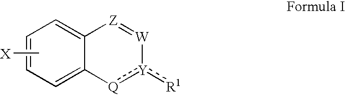
or a pharmaceutically acceptable base or acid addition salt, hydrate, stereoisomer, or mixture thereof, wherein
X is one or more halogen radicals;
Q is NH or N;
W is CR², CHR², NR³, or CH═COH;
Y is C or CH;
Z is C═O, SO₂, COH, CHOH, or NHR⁴;
R¹ is CO₂H or oxo (═O);
R² is H, C(═O)—C₁-C₆ alkyl, C(═O)O—C₁-C₆ alkyl, or C(═O)—C₃-C₈ cycloalkyl;
R³ is optionally substituted C₃-C₁₀ aryl; and
R⁴ is 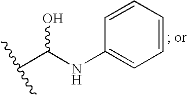
R² and R¹ combine with the carbons to which they are attached to form a 6 membered heterocycle that is optionally substituted at one or more of the heteroatoms with C₃-C₁₀ aryl, wherein the aryl may be substituted with one or more of C₁-C₆ alkyl or —O—C₁-C₆ alkyl, and the heteroatoms in the heterocyclic ring are one or more nitrogen atoms.

60. A method for inhibiting activity of a Parkinson's disease-associated LRRK2 mutant protein in a neuronal cell, the method comprising contacting the cell with a compound of a compound of Formula II: 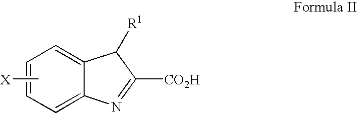
or a pharmaceutically acceptable base or acid addition salt, hydrate, stereoisomer, or mixture thereof, wherein
X is one or more halogen radicals;
R¹ is (CH₂)ₙ—CO₂H, or CH═CHC(═O)NHR²; and
R² is C₃-C₁₀ aryl optionally substituted with one or more of C₁-C₆ alkyl, —O—C₁-C₆ alkyl, or halogen; and
n is 0, 1, 2, 3, 4, 5, or 6.

61. A method for increasing axonal length, axonal branching, or both in a neuronal cell, the method comprising contacting the cell with an N-methyl-D-aspartic acid (NMDA) receptor antagonist, an antioxidant or both.

62. The method of claim 61, wherein the NMDA receptor antagonist is 2-amino-5-phosphonopentanoate (AP-5), MK-801, L-701,324, kynurenine, 1-aminocyclobutane carboxylic acid (ACBC), hydroquinone, or a structural analog thereof.

63. The method of claim 61, wherein the antioxidant is glutathione, hydroquinone, or a structural analog thereof.

64. A method for increasing axonal length, axonal branching, or both in a neuronal cell, the method comprising contacting a neuronal cell with a compound of Formula I: 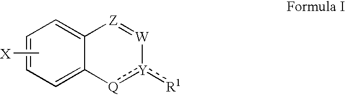
or a pharmaceutically acceptable base or acid addition salt, hydrate, stereoisomer, or mixture thereof, wherein
X is one or more halogen radicals;
Q is NH or N;
W is CR², CHR², NR³, or CH═COH;
Y is C or CH;
Z is C═O, SO₂, COH, CHOH, or NHR⁴;
R¹ is CO₂H or oxo (═O);
R² is H, C(═O)—C₁-C₆ alkyl, C(═O)O—C₁-C₆ alkyl, or C(═O)—C₃-C₈ cycloalkyl;
R³ is optionally substituted C₃-C₁₀ aryl; and
R⁴ is 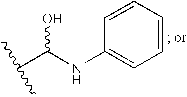
R² and R¹ combine with the carbons to which they are attached to form a 6 membered heterocycle that is optionally substituted at one or more of the heteroatoms with C₃-C₁₀ aryl, wherein the aryl may be substituted with one or more of C₁-C₆ alkyl or —O—C₁-C₆ alkyl, and the heteroatoms in the heterocyclic ring are one or more nitrogen atoms.

65. A method for increasing axonal length, axonal branching, or both in a neuronal cell, the method comprising contacting a neuronal cell with a compound of a compound of Formula II: 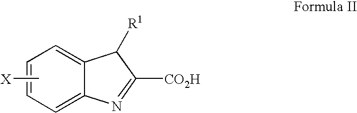
or a pharmaceutically acceptable base or acid addition salt, hydrate, stereoisomer, or mixture thereof, wherein
X is one or more halogen radicals;
R¹ is (CH₂)ₙ—CO₂H, or CH═CHC(═O)NHR²; and
R² is C₃-C₁₀ aryl optionally substituted with one or more of C₁-C₆ alkyl, —O—C₁-C₆ alkyl, or halogen; and
n is 0, 1, 2, 3, 4, 5, or 6.
